(19) United States
(12) Patent Application Publication
(10) Pub. No.: US 2024/0158808 A1
(43) Pub. Date: May 16, 2024

(54) GENE THERAPY FOR DENT DISEASE

(71) Applicant: Wake Forest University Health Sciences, Winston-Salem, NC (US)

(72) Inventors: Baisong Lu, Winston-Salem, NC (US); Anthony Atala, Winston-Salem, NC (US)

(21) Appl. No.: 18/289,875

(22) PCT Filed: May 20, 2022
(86) PCT No.: PCT/US22/30264

§ 371 Date: Nov. 7, 2023

Related U.S. Application Data
(60) Provisional application No. 63/193,212, filed on May 26, 2021.

(51) Int. Cl. C12N 15/86; A01K 67/0276; A61K 48/00; A61P 13/12; C07K 14/47

(52) U.S. Cl. CPC C12N 15/86; A01K 67/0276; A61K 48/0058; A61K 48/0075; A61P 13/12; C07K 14/47; A01K 2217/075; A01K 2227/105; A01K 2267/03; C12N 2740/16043; C12N 2800/22; C12N 2830/008

(57) ABSTRACT

The present invention includes methods and compositions useful for the treatment of Dent's disease in a subject in need thereof. The invention of the present disclosure also includes a mouse model useful for the study of Dent's disease.

### CROSS-REFERENCE TO RELATED APPLICATION

The present application is entitled to priority under 35 U.S.C. § 119(e) to U.S. Provisional Patent Application No. 63/193,212, filed May 26, 2021, which is hereby incorporated by reference in its entirety herein.

### SEQUENCE LISTING

The ASCII text file named “205286_7010WO1SequenceListing” created on May 20, 2022, comprising 29 Kbytes, is hereby incorporated by reference in its entirety.

### BACKGROUND OF THE INVENTION

Dent disease is a chronic kidney disorder characterized by abnormally high amounts of protein and excess calcium in the urine. Dent disease is caused by genetic mutations that reduce the ability of cells of the proximal renal tubule to reabsorb nutrients, water, and other substances that have been filtered from the bloodstream. Clinical symptoms of Dent disease appear in childhood and worsen over time. The kidney dysfunction causing Dent disease progressively damages kidney cells and eventually causes a range of symptoms from calcifications in the kidney tissue, kidney stones, abdominal pain, repeated urinary tract infections, chronic kidney disease, and kidney failure.

Genetically, Dent disease is caused by loss-of-function mutations in either the CLCN5 or OCRL1 genes, which separate the disease into two types. Type 1 Dent disease is characterized by mutations in the CLCN5 gene, while type 2 Dent disease is associated with mutations OCRL1. Both genes are X-linked and recessive, resulting in the majority of patients being male, though females can be asymptomatic “carriers” and can suffer mild hypercalciuria due to random X-chromosome inactivation. Type 1 Dent disease is more common with around 60% of total cases with Type 2 being 15% of cases and the remaining 25% being of unknown etiology. Type 2 Dent disease is often associated with mild intellectual disability, hypotonia, and mild cataract. Currently there are only supportive treatments for Dent disease and none of them target the root of the disease. Severe cases are often treated by kidney transplant, though such a strategy requires identifying a compatible donor, invasive surgery, and life-long immune suppression to delay rejection.

Thus there is a need for treatments that correct the mutated genes responsible for Dent disease in order to restore normal kidney function. The current invention addresses these needs.

### SUMMARY OF THE INVENTION

As described herein, the present invention relates to methods and compositions useful for the treatment of type 1 Dent disease in a subject in need thereof. The invention of the present disclosure also includes a mouse model useful for the study of Dent's disease.

As such, in one aspect, the invention includes a method for treating Dent disease in a subject in need thereof, the method comprising administering to the subject an effective amount of a nucleic acid vector encoding a CLCN5 protein, thereby treating the disease.

In certain embodiments, the nucleic acid vector is a lentiviral vector.

In certain embodiment, the nucleic acid vector is operably linked to a promoter that drives the expression of the CLCN5 protein.

In certain embodiments, the promoter is a constitutive promoter.

In certain embodiments, the promoter is an EF-1α promoter.

In certain embodiments, the promoter is a tissue-specific promoter.

In certain embodiments, the tissue-specific promoter is specific for renal tubule proximal cells.

In certain embodiments, the tissue specific promoter is selected from the group consisting of Npt2a and Sgt12.

In certain embodiments, the lentiviral vector is encoded by the nucleic acid sequence set forth in SEQ ID NO. 1.

In certain embodiments, the administration is delivered locally to the kidney.

In certain embodiments, the local kidney administration is delivered by retrograde ureteral injection.

In another aspect, the invention includes a method for correcting a mutation in the CLCN5 gene in a cell, said method comprising contacting the cell with a nucleic acid vector encoding a functional CLCN5 protein.

In certain embodiments, the nucleic acid vector is a lentiviral vector.

In certain embodiments, the nucleic acid vector is operably linked to a promoter that drives expression of the CLCN5 protein.

In certain embodiments, the promoter is a constitutive promoter.

In certain embodiments, the promoter is an EF-1α promoter.

In certain embodiments, the promoter is a tissue-specific promoter.

In certain embodiments, the tissue-specific promoter is specific for renal tubule proximal cells.

In certain embodiments, the tissue specific promoter is selected from the group consisting of Npt2a and Sgt12.

In certain preferred embodiments, the lentiviral vector is encoded by the nucleic acid sequence set forth in SEQ ID NO: 1.

In another aspect, the invention provides a pharmaceutical composition comprising a nucleic acid vector encoding a CLCN5 protein and a pharmaceutically acceptable carrier.

In certain embodiments, the nucleic acid vector is a lentiviral vector.

In certain embodiments, the lentiviral vector is encoded by a nucleic acid sequence set forth in SEQ ID NO: 1.

In another aspect, the invention includes a mouse model of type 1 Dent disease, wherein the mouse comprises one or more mutation in the CLCN5 gene in the mouse.

In certain embodiments, the one or more mutations is a deletion.

In certain embodiments, the deletion affects exon 3, exon 4, exon 5, exon 6, exon 7, exon 8, exon 9, exon 10, and exon 11 of the CLCN5 gene.

In certain embodiments, the one or more CLCN5 mutations result in a non-functional CLCN5 protein.

In certain embodiments, the breeding of experimental animals involves a sire and dam being of different strains.

In certain embodiments, the dam is a heterozygous for the CLCN5 mutation and the sire is wildtype.

In certain embodiments, the sire is of the FVB background.

In certain embodiments, the dam is of the C57BL/6 background.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIGS. 1A-1B are diagrams showing the mutational landscape of the CLCN5 gene in Dent disease. FIG. 1A is a diagram of the CLCN5 gene showing the location and type of known mutations. FIG. 1B is a chart showing the frequency of each type of mutation.

FIGS. 2A-2B are diagrams displaying the strategy of creating a Dent disease mouse model via the deletion of CLCN5. FIG. 2A shows the locations of the ends of the deleted area (arrows) which spans exons 3-11. FIG. 2B is a sequence of the completed mutant showing the successful deletion of the targeted area.

FIGS. 3A-3B illustrate the breeding strategy required to generate CLCN5 knockout mice. FIG. 3A illustrates that the expected Mendelian ratio of normal and knockout mice is 50/50, however when a C57BL/6 female carrier is bred to a normal male of the same strain, much fewer than expected knockout male pups are born, suggesting embryonic lethality. FIG. 3B shows the mixed C57BL/6 and FVB background breeding strategy required to obtain expected ratios of knockout, heterozygous (carrier), and wildtype pups.

FIG. 4 illustrates that CLCN5 knockout mice do not produce detectable levels of CLCN5 mRNA or protein.

FIG. 5 illustrates that CLCN5 knockout mice secrete dramatically more albumin in their urine than wildtype mice as assayed by SDS-PAGE. Three concentrations (50 μg, 100 μg, and 200 μg) from two knockout and two wildtype mice were separated on the gel. Box indicates a band (possibly albumin) at −66 kDa.

FIG. 6 illustrates a study confirming that urine protein secretion is higher in CLCN5 mutant mice, as assayed by Western blotting for albumin (left) and vitamin D binding protein (right).

FIG. 7 is a diagram illustrating the design of a lentiviral vector for hCLCN5 expression.

FIG. 8 illustrates that the CLCN5 lentiviral vector is able to induce CLCN5 expression in transduced cells, as measured by RT-qPCR (left) and Western blotting (right).

FIGS. 9A-9B illustrates the delivery of CLCN5 lentiviral vectors via retrograde ureteral injection. FIG. 9A is a diagram of retrograde ureteral injection (left) and a micrograph of the successfully located ureter and kidney during the injection procedure.

FIG. 9B are fluorescence micrographs of kidney tissue from mice injected one week previously with a GFP-expressing lentivirus (right) or untreated control mice (left).

FIG. 10 is a diagram of the setup of an in vivo study treating CLCN5 knockout mice with CLCN5-expressing lentiviral vectors delivered via retrograde ureteral injection.

FIG. 11 illustrates that treatment of mutant mice with the CLCN5 lentivirus greatly reduces urine protein, as assessed by SDS-PAGE.

FIG. 12 illustrates the reduction of specific urine proteins in lentivirus-treated knockout mice. Studies assessed albumin (left) or vitamin D binding protein (DBP, right).

FIG. 13 illustrates the reduction in CC16 protein in lentivirus-treated mice.

FIG. 14 illustrates that the lentivirus therapy-induced reduction in urine protein in mutant mice was durable out to two months following injection as assessed by SDS-Page (left), while untreated mutants did not demonstrate any reduction in protein levels (right).

FIG. 15 illustrates the volume (top) and levels of protein (middle) and calcium ions (bottom) in the urine of CLCN5 lentivirus-treated knockout mice, untreated controls, or mice receiving a GFP control lentivirus.

FIGS. 16A-16E illustrate the generation and characterization of CLCN5 knockout mice. FIG. 16A. Gene structure of mouse CLCN5 and the sgRNAs used for deleting the 26 kilo bp region. FIG. 16B. Confirming the lack of CLCN5 mRNA expression in the kidney of mutant mice by RT-PCR. Two normal and two mutant male mice were analyzed. RT-: PCR products using templates of RNA from normal kidney without reverse transcription. Primers were specific to mouse CLCN5 cDNA. FIG. 16C. Western blotting analysis of CLCN5 protein in kidney tissues of wild type and mutant mice. FIG. 16D. SDS-PAGE analysis of urine proteins of wild type and mutant males. * indicates the 61 kDa protein band observed in urine samples from mutant mice but not those from wild type mice. FIG. 16E. Western blotting analysis of urine protein from normal and mutant mice. The sample order for CC10 was re-arranged to match those of Alb and DBP. Alb: albumin; DBP: vitamine D-binding protein; CC10: Clara Cells 10 KDa Secretory Protein. For (FIGS. 16D and 16E), equal volumes of urine samples were analyzed and each lane contained urine sample from a different mouse.

FIGS. 17A-17E illustrate the expression of human CLCN5 in the kidney of mutant mice. FIG. 17A. Components of the human CLCN5-expressing lentiviral vector. FIG. 17B. Detecting mRNA expression in HEK293T cells by RT-qPCR. CLCN5- and GFP-expressing lentiviral vectors (10 ng p24) were transduced into 2.5×104 HEK293T cells. Forty-eight hours after transduction, CLCN5 expression was detected by qRT-PCR with primers hCLCN5-F and hCLCN5-R (see Table 1 for sequences). The primers were specific for the codon-optimized human CLCN5 mRNA expressed from the lentiviral vectors and could not detect the endogenous CLCN5 mRNA. *** indicates p<0.0001 (t-tests). FIG. 17C. Western blotting detection of CLCN5 protein in transduced kidney proximal tubule cells. CLCN5-expressing lentiviral vectors (28 ng p24) were transduced into 2.5×105 kidney proximal tubule cells isolated from wild type and mutant mice. Western blotting was performed 72 hours after transduction. FIG. 17D. Detecting CLCN5 protein by Western blotting 2 weeks after delivering CLCN5 LV into the kidneys of mutant mice. FIG. 17E. Detecting CLCN5 protein expression by immunofluorescence 2 weeks after delivering CLCN5 LV into the kidneys of mutant mice. FITC- and Alex-594-conjugated secondary antibodies were used to detect CLCN5 in wild type and mutant mice respectively.

FIGS. 18A-18E illustrate the therapeutic effects of CLCN5 gene therapy. FIG. 18A. Immunofluorescent analysis of megalin expression in mutant mice with and without CLCN5 LV delivery. Nuclei were stained with 4′, 6-diamidino-2-phenylindole (DAPI) and shown in blue. FIG. 18B. Quantitative analysis of tubular mean fluorescent intensity by ImageJ. FIG. 18C. SDS-PAGE analysis of urine proteins after delivering lentiviral vectors into both kidneys of mutant mice. FIG. 18D. Western blotting detection of urine marker proteins before and after CLCN5 LV injection into the left kidney of mutant mice. FIG. 18E. Western blotting detection of urine marker proteins before and after gene delivery into both kidneys of mutant mice. For (FIGS. 18C-18E), urine samples were collected one month after viral vector injection. Equal urine volume was analyzed for each sample. Each lane contained sample from a different mouse.

FIGS. 19A-19B illustrate that therapeutic effects lasted for up to 4 months following gene delivery. FIG. 19A. Diuresis, urine protein and urine calcium levels at various time points following gene therapy. Group size was indicated by n. ***indicates P<0.0001 when mice treated for both kidneys were compared with ZsGreen LV treated mice (two-tailed t-tests). FIG. 19B. Western blotting analysis of urine marker proteins at various time points following CLCN5 gene delivery. Equal volume of urine samples from one kidney treated mice were loaded in each lane.

FIGS. 20A-20F illustrate that delivery of second dose of LV suggests involvement of immune responses. FIG. 20A. Scheme of the experiment. Solid triangles indicate the times for therapeutic effect assessment. FIG. 20B. SDS-PAGE analysis of urine proteins. All mice were male mutants. Mouse No. 6 was a naïve mouse receiving the first dose of viral vector and mice 1-5 were male mutant mice received a second CLCN5 LV dose 5 months after receiving the first dose. FIG. 20B: before viral injection; FIG. 20A: after viral injection. FIG. 20C. Effects of first and second dose of viral injection on diuresis (left), urine protein (middle) and urine calcium (right) excretion. Data were from the same five mice receiving the first and second dose. *: P<0.05 (Bonferroni posttests following two-way ANOVA). FIG. 20D. Detecting vector genomic DNA after first and second vector injection using qPCR. FIG. 20E. Detecting hCLCN5 mRNA expression after first and second vector injection using RT-qPCR. FIG. 20F. Detecting CLCN5 protein expression after first and second vector injection using Western blotting. The same mice were analyzed in panels FIG. 20B, FIG. 20D, FIG. 20E and FIG. 20F.

FIG. 21 illustrates the confirming CLCN5 gene knockout by DNA sequencing. Sequences above the horizontal arrows were deleted for the three founder females (No. 6, 20 and 34). The sgRNA target sequences (underlined) in intron 2 and exon 12 are shown. PAMs are in green. A reverse primer in exon 12 was used for sequencing. The junctions between intron 2 and exon 12 are indicated by a vertical arrow.

FIG. 22 illustrates that CLCN5 mutant males were obtained less than expected. *** indicates p<0.0001 in Fisher's exact test.

FIG. 23 illustrates the reduction of urine proteins after delivery of CLCN5-expressing lentiviral vectors into the left kidney of mutant mice. Urine samples were collected one month after viral vector injection. The same urine volume was analyzed for each sample.

FIG. 24 illustrates that urine protein levels returned to pre-treatment level 4 months after gene delivery. The same urine volume was analyzed for each sample. B: before treatment; 1 m-4 m: 1, 2, 3 or 4 months after gene delivery. Samples from two representative mice are shown.

FIGS. 25A-25B illustrate the generation and characterization of Clcn5 knockout mice. FIG. 25A. Comparing urine volume, urine calcium and urine protein of female mice. Wild type, heterozygous and homozygous mutant mice were 81 days old. *, ** and *** indicate p<0.05, p<0.01 and p<0.001 between the indicated groups (Tukey's Multiple Comparison Test following one-way ANOVA). FIG. 25B. Comparing urine volume, urine calcium and urine protein of male mice. Urine samples were collected from mice of 2-2.5 months. *** indicates p<0.0001 between wild type and mutant mice (two-tailed unpaired t-tests).

FIGS. 26A-26B illustrate the delivery of LV vector to mouse kidney by retrograde ureter injection. FIG. 26A. Detecting GFP protein expression in mouse kidney 2 weeks after GFP LV delivery by retrograde ureter injection. The mouse was 6-month-old, wild type, and received GFP LV injection in both kidneys. GFP expression was detected by immunofluorescence (shown in red). Insert was an enlarged view of a GFP-positive tubule. Nuclei were stained by 4′, 6-diamidino-2-phenylindole (DAPI, shown in blue). FIG. 26B. Detecting GFP LV DNA in various organs by qPCR two weeks after GFP LV delivery. Genomic DNA samples isolated from different organs were used as template in qPCR to detect GFP DNA. Mouse No. 1 was the same mouse shown in FIG. 26A. Mice No. 2, 3 and 4 were male Clcn5 mutant mice receiving GFP LV injection 10 months following CLCN5 LV injection. All mice were euthanized two weeks after GFP LV injection. The dashed line indicates detection limit.

FIGS. 27A-27C illustrate CLCN5 LV restored CLCN5 expression in the kidneys of mutant mice. FIG. 27A. Detecting CLCN5 protein by immunofluorescence in wild type kidney. The insert shows the relative weak CLCN5 expression in the glomeruli marked by an asterisk. FIG. 27B. Undetectable CLCN5 protein in the kidney of mutant mice without CLCN5 LV injection. FIG. 27C. Detecting CLCN5 protein in the kidneys of mutant mice 2 weeks following CLCN5 LV injection. The two half images were from two injected kidneys with different CLCN5 expression levels.

FIGS. 28A-28C illustrate the therapeutic effects of CLCN5 LV gene therapy. FIG. 28A. Effects of CLCN5 LV delivery on diuresis of mutant mice. FIG. 28B. Effects of CLCN5 LV delivery on urine calcium of mutant mice. FIG. 28C. Effects of CLCN5 LV delivery on urine protein of mutant mice. For (FIG. 28A-FIG. 28C), all mutant mice received 280 ng p24 of CLCN5 or ZsGreen LV to the left kidney at the age of 87 days. Data from each mouse were presented. The first datum point showed the time of LV injection and the pre-treatment urine parameters from urine samples collected 37 days before LV injection. Post-treatment data showed urine parameters from urine samples collected at the indicated ages. A dashed line indicates values of wild type male mice presented in previous studies presented herein. ***indicates p<0.001 compared with pretreatment values (Tukey's Multiple Comparison Test following one-way ANOVA).

FIG. 29A-29C illustrate Therapeutic effects of delivering CLCN5 LV into both kidneys. FIG. 29A. Effects of CLCN5 LV delivery on diuresis of mutant mice. Age-matched mutant mice were injected with CLCN5 LV or ZsGreen LV in both kidneys. For visibility, data from 3 of 5 pairs were presented here and data from the other two pairs were presented in FIG. 33. FIG. 29B. Effects of CLCN5 LV delivery on urine calcium of mutant mice. FIG. 29C. Effects of CLCN5 LV delivery on urine protein of mutant mice. All mutant mice received 280 ng p24 of CLCN5 or ZsGreen LV to both kidneys at ages of the first data points. Data from each mouse were presented. Pre-treatment urine samples were collected 27 days before LV injection. The age of the first datum point for each mouse was the age of injection. Post-treatment urine samples were collected at the indicated ages. A dashed line indicates values of wild type male mice presented in previous studies of the present disclosure.

FIG. 30 illustrates DNA sequencing analysis of predicted off-targets in Clcn5 gene knockout mice. The protospacer adjacent motifs (or the reverse complementary sequences) were underlined with red lines and the target sequences were underlined with black lines. Off 1, Off 2 and Off 3 were off-targets for sgRNA 1, sgRNA 2 and sgRNA 3 respectively. The last image was the only off-target on protein coding gene. The four off-targets on X chromosome were also labeled.

FIGS. 31A-31C illustrate the effects of delivering CLCN5 LV to the left kidney. FIG. 31A. Urine volume. FIG. 31B. Urine calcium. FIG. 31C. Urine protein. CLCN5 LV (280 ng p24) injection was performed on the day of the first datum point for each mouse. The urine was collected 37 days before LV injection. The second, third and fourth data points showed the actual time when the urine samples were collected.

FIGS. 32A-32C illustrate that age did not greatly affect the urine parameters of mutant mice. FIG. 32A. Urine volume. FIG. 32B. Urine calcium. FIG. 32C. Urine protein. Each datum point was from a different male mutant mouse. The dashed lines show the 95% confidence intervals.

FIG. 33 illustrates that CLCN5 gene therapy on diuresis. Two of the 5 age-matched pairs were presented here for visibility. The other three pairs were shown in FIG. 6A. Both kidneys were treated.

FIGS. 34A-34C illustrate the effects of delivering CLCN5 LV to both kidneys. FIG. 34A. Urine volume. FIG. 34B. Urine calcium. FIG. 34C. Urine protein. CLCN5 LV (280 ng p24/kidney) injection was performed on the day of the first datum point for each mouse. The urine was collected 7 days before LV injection. The second, third, fourth and fifth data points showed the actual age when the urine samples were collected.

FIG. 35 depicts detecting GFP protein by immunofluorescence in mouse kidney with and without CLCN5 LV injection. Naïve mouse was a 6-month wild type mouse receiving GFP LV injection without CLCN5 LV pre-injection. The other three mice (CLCN5-LV, GFP-LV, No. 2-4) were mutant mice that received GFP LV injection 10 months following CLCN5 LV injection. The mice were euthanized 2 weeks after GFP LV injection.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a”, “an”, and “the” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A “biomarker” or “marker” as used herein generally refers to a nucleic acid molecule, clinical indicator, protein, or other analyte that is associated with a disease. In certain embodiments, a nucleic acid biomarker is indicative of the presence in a sample of a pathogenic organism, including but not limited to, viruses, viroids, bacteria, fungi, helminths, and protozoa. In various embodiments, a marker is differentially present in a biological sample obtained from a subject having or at risk of developing a disease (e.g., an infectious disease) relative to a reference. A marker is differentially present if the mean or median level of the biomarker present in the sample is statistically different from the level present in a reference. A reference level may be, for example, the level present in an environmental sample obtained from a clean or uncontaminated source. A reference level may be, for example, the level present in a sample obtained from a healthy control subject or the level obtained from the subject at an earlier timepoint, i.e., prior to treatment. Common tests for statistical significance include, among others, t-test, ANOVA, Kruskal-Wallis, Wilcoxon, Mann-Whitney and odds ratio. Biomarkers, alone or in combination, provide measures of relative likelihood that a subject belongs to a phenotypic status of interest. The differential presence of a marker of the invention in a subject sample can be useful in characterizing the subject as having or at risk of developing a disease (e.g., an infectious disease), for determining the prognosis of the subject, for evaluating therapeutic efficacy, or for selecting a treatment regimen.

By “agent” is meant any nucleic acid molecule, small molecule chemical compound, antibody, or polypeptide, or fragments thereof.

By “alteration” or “change” is meant an increase or decrease. An alteration may be by as little as 1%, 2%, 3%, 4%, 5%, 10%, 20%, 30%, or by 40%, 50%, 60%, or even by as much as 70%, 75%, 80%, 90%, or 100%.

By “biologic sample” is meant any tissue, cell, fluid, or other material derived from an organism.

By “capture reagent” is meant a reagent that specifically binds a nucleic acid molecule or polypeptide to select or isolate the nucleic acid molecule or polypeptide.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

By “detectable moiety” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

A “disease” is a state of health of an animal wherein the animal cannot maintain homeostasis, and wherein if the disease is not ameliorated then the animal's health continues to deteriorate. In contrast, a “disorder” in an animal is a state of health in which the animal can maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

By “fragment” is meant a portion of a nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 5, 10, 15, 20, 30, 40, 50, 60, 70, 80, 90, or 100 nucleotides.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Hybridization” means hydrogen bonding, which may be Watson-Crick, Hoogsteen or reversed Hoogsteen hydrogen bonding, between complementary nucleobases. For example, adenine and thymine are complementary nucleotides that pair through the formation of hydrogen bonds.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “marker profile” is meant a characterization of the signal, level, expression or expression level of two or more markers (e.g., polynucleotides).

By the term “microbe” is meant any and all organisms classed within the commonly used term “microbiology,” including but not limited to, bacteria, viruses, fungi and parasites.

By the term “microarray” is meant a collection of nucleic acid probes immobilized on a substrate. As used herein, the term “nucleic acid” refers to deoxyribonucleotides, ribonucleotides, or modified nucleotides, and polymers thereof in single- or double-stranded form. The term encompasses nucleic acids containing known nucleotide analogs or modified backbone residues or linkages, which are synthetic, naturally occurring, and non-naturally occurring. Nucleic acid molecules useful in the methods of the invention include any nucleic acid molecule that specifically binds a target nucleic acid (e.g., a nucleic acid biomarker). Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

“Parenteral” administration of an immunogenic composition includes, e.g., subcutaneous (s.c.), intravenous (i.v.), intramuscular (i.m.), or intrasternal injection, or infusion techniques.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By “reference” is meant a standard of comparison. As is apparent to one skilled in the art, an appropriate reference is where an element is changed in order to determine the effect of the element. In one embodiment, the level of a target nucleic acid molecule present in a sample may be compared to the level of the target nucleic acid molecule present in a clean or uncontaminated sample. For example, the level of a target nucleic acid molecule present in a sample may be compared to the level of the target nucleic acid molecule present in a corresponding healthy cell or tissue or in a diseased cell or tissue (e.g., a cell or tissue derived from a subject having a disease, disorder, or condition).

As used herein, the term “sample” includes a biologic sample such as any tissue, cell, fluid, or other material derived from an organism.

By “specifically binds” is meant a compound (e.g., nucleic acid probe or primer) that recognizes and binds a molecule (e.g., a nucleic acid biomarker), but which does not substantially recognize and bind other molecules in a sample, for example, a biological sample.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95%, 96%, 97%, 98%, or even 99% or more identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e′ and e⁻¹⁰⁰ indicating a closely related sequence.

By the term “substantially microbial hybridization signature” is a relative term and means a hybridization signature that indicates the presence of more microbes in a tumor sample than in a reference sample. By the term “substantially not a microbial hybridization signature” is a relative term and means a hybridization signature that indicates the presence of less microbes in a reference sample than in a tumor sample.

By “subject” is meant a mammal, including, but not limited to, a human or non-human mammal, such as a bovine, equine, canine, ovine, feline, mouse, or monkey. The term “subject” may refer to an animal, which is the object of treatment, observation, or experiment (e.g., a patient).

By “target nucleic acid molecule” is meant a polynucleotide to be analyzed. Such polynucleotide may be a sense or antisense strand of the target sequence. The term “target nucleic acid molecule” also refers to amplicons of the original target sequence. In various embodiments, the target nucleic acid molecule is one or more nucleic acid biomarkers.

A “target site” or “target sequence” refers to a genoinic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

The terms “Dent disease” or “Dent's disease” as used herein refer to an X-linked renal syndrome of low molecular weight proteinuria, hypercalciuria, aminoaciduria, and hypophosphatemia caused by mutational defects in the genes encoding CLCN5 and/or OCRL1 proteins resulting in the partial or complete loss of function of these genes. Loss of CLCN5 is associated with Type 1 Dent disease, while loss of OCRL1 is associated with Type 2 Dent disease.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based on the observations described herein that the genetic abnormalities that result in the clinical disorders known as Dent disease can be treated by providing nucleic acid vectors encoding functional replacements for the abnormal genes. The invention also includes a mouse model of Dent disease, wherein the expression of a Dent disease-related gene is knocked-out in mutant mice, said model being useful for the study of Dent disease and the development of therapies for the disease. As such, in one aspect, the invention includes a method for treating Dent disease in a subject in need thereof, said method comprising administering to the subject an effective amount of a nucleic acid vector encoding a CLCN5 protein, thereby treating the disease. In another aspect, the invention of the current disclosure includes a method for correcting a mutation in the CLCN5 gene in a cell, said method comprising contacting the cell with a nucleic acid vector encoding a functional CLCN5 protein.

### Dent Disease

Dent disease is a kidney disorder characterized by the secretion of large amounts of small proteins and calcium ions into the urine, kidney calcifications, kidney stones, and chronic kidney disease. Advanced forms of the disease can result in kidney failures. Dent disease is X-linked, resulting in most patients being male; however, heterozygous females may suffer milder forms of the disease presumably due to random X inactivation in kidney tissues. Symptoms of Dent disease usually appear in childhood; however, mild cases may remain undetected until adulthood. In some cases, the disorder will progressively worsen over time leading to chronic kidney disease and renal failure, typically by 30 to 50 years of age.

Dent disease is subdivided into two types. Type 1 Dent disease is characterized by solely by the aforementioned kidney symptoms, while Type 2 Dent disease is characterized by the same kidney symptoms usually accompanied by other developmental disorders including mild intellectual disability, eye involvement or diminished muscle tone (hypotonia). Type 1 Dent disease is caused by mutations in the CLCN5 gene, while Type 2 Dent disease is caused by mutations in the OCRL1 gene, which are both located on the X chromosome. These mutations may be inherited or can occur randomly with no previous family history.

The CLCN5 gene encodes a voltage-gated chloride ion channel in the chloride channel (CLC) family. CLCN5 is most highly expressed in renal proximal tubule cells, which normally reabsorb proteins passing the glomerular filter. A number of different mutations to CLCN5 have been observed in relation to Dent disease, with all of them resulting in the loss of CLCN5 protein expression, or the expression of non-functional protein.

Current treatments for Dent disease involve supportive care for specific symptoms in individuals that do not address the underlying genetic abnormalities. Thus in certain embodiments, the current invention includes methods for treating Dent disease that comprise providing functional copies of the CLCN5 gene and CLCN5 protein to affected tissues. In certain embodiments, the CLCN5 protein is delivered by way of a nucleic acid vector encoding the CLCN5 protein.

### Gene Transfer Systems and Lentiviral Vectors

Gene transfer systems, such as those described in the present invention, depend upon a vector or vector system to shuttle the genetic constructs into target cells. Methods of introducing a nucleic acid into target cells and tissues include physical, biological and chemical methods. Physical methods for introducing a polynucleotide, such as RNA, into a target cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

Chemical means for introducing a polynucleotide into a target cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., (1991) Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Biological methods for introducing a polynucleotide of interest into a host cell include the use of DNA and RNA vectors. Viral vectors, and especially lentiviral vectors, have become the most widely used method for inserting genes into mammalian, e.g., human cells. Viral vectors can be derived from lentivirus, poxviruses, herpes simplex virus I, adenoviruses and adeno-associated viruses, and the like. See, for example, U.S. Pat. Nos. 5,350,674 and 5,585,362.

In certain embodiments, the invention includes nucleic acid vectors which encode a CLCN5 protein. In certain embodiments, the nucleic acid vectors are lentiviral vectors. Lentiviral vectors are useful for transducing a target cell with a nucleotide payload. Once within the cell, the RNA genome of the vector is reverse transcribed into DNA and integrated into the genome of the target cell. Lentiviral vectors are part of a larger group of retroviral vectors. A detailed list of lentiviruses may be found in (Coffin et al. (1997) “Retroviruses” Cold Spring Harbor Laboratory Press. pp 758-763).

Lentiviruses can be divided into primate and non-primate groups. Examples of primate lentiviruses include but are not limited to: human immunodeficiency virus (HIV), and simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV). Lentiviruses differ from other members of the retrovirus family in that lentiviruses have the capability to infect both dividing and non-dividing cells, which make them attractive vectors for in vivo gene therapies (Lewis et al (1992) EMBO J 11(8):3053-3058) and Lewis and Emerman (1994) J Virol 68 (1):510-516).

The basic structure of retrovirus and lentivirus genomes share many common features such as a 5′ LTR and a 3′ LTR, between or within which are located a packaging signal to enable the genome to be packaged, a primer binding site, integration sites to enable integration into a host cell genome and gag, pol and env genes encoding the viral particle components.

In the provirus, or the nucleic acid molecule of the vector that integrates into the target cell genome, the viral and payload genes are flanked at both ends by regions called long terminal repeats (LTRs). The LTRs are responsible for proviral integration, and transcription. LTRs also serve as enhancer-promoter sequences and can control the expression of the viral and payload genes.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different viruses.

In order to render lentiviral vectors incapable of replicating in target cells, most vectors have deletions or mutation in the gag, pol and env genes which render them absent or non-functional. In certain embodiments, the lentiviral vectors of the current invention may comprise one or more of these modifications which make the viral vector replication-defective.

In certain embodiments, the lentiviral vectors of the current invention may be self-inactivating lentiviral vectors. Self-inactivating retroviral vectors comprise deletions of the transcriptional enhancers and/or promoters in the U3 and U5 regions of the LTRs. However, any promoters contained within the transduced DNA sequence between the LTRs in such vectors remains transcriptionally active. This strategy has been employed to eliminate effects of the enhancers and promoters in the viral LTRs on transcription from internally placed genes. Such effects include increased transcription (Jolly et al (1983) Nucleic Acids Res. 11:1855-1872) or suppression of transcription (Emerman and Temin (1984) Cell 39:449-467). This strategy can also be used to eliminate downstream transcription from the 3′ LTR into genomic DNA (Herman and Coffin (1987) Science 236:845-848). Such modifications are particularly helpful in lentiviral vectors used for human gene therapy where activation of an endogenous oncogenes is to be avoided.

Regardless of the method used to introduce the nucleic acid into the cell, a variety of assays may be performed to confirm the presence of the nucleic acid in the cell. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify payload proteins falling within the scope of the invention.

### Methods of Treatment

In certain embodiments, the nucleic acid vector described herein is a lentiviral vector. In certain embodiments, the nucleic acid vector may be included in a pharmaceutical composition useful for treating Dent disease in a subject in need thereof. The composition may include a pharmaceutical composition and further include a pharmaceutically acceptable carrier. A therapeutically effective amount of the pharmaceutical composition comprising the nucleic acid vector may be administered.

In one aspect, the present invention includes a method for treating Dent disease in a subject in need thereof, the method comprising administering to the subject an effective amount of a nucleic acid vector encoding a CLCN5 protein, thereby treating the disease. In another aspect, the invention includes a method for correcting a mutation in the CLCN5 gene in a cell, the method comprising contacting the cell with a nucleic acid vector encoding a functional CLCN5 protein. In certain embodiments of the above aspects, the nucleic acid vector is a lentiviral vector.

In certain embodiments, the lentiviral vectors and compositions of the current invention are delivered locally to the target tissue, including the various parts of the kidney. In certain embodiments, the delivery of the CLCN5 protein is most beneficial when targeted to cells whose normal function depends on the expression of CLCN5 protein. In the kidney, these cells include but are not limited to epithelial cells lining the proximal tubules and the thick ascending limbs of the Henle loop, and in the intercalated cells of the collecting ducts. In certain embodiments, local administration of the lentiviral vectors or compositions of the invention to the kidney is achieved via retrograde ureteral injection. In this way, the lentiviral particles have direct contact with the target tissue via the lumen of the renal tubules and ducts. In certain embodiments, the retrograde injection is followed temporary ligation or partial ligation of the ureter which prevent flushing of the lentiviral particles out of the kidney tissue before they can contact target cells.

### Mouse Models of Dent Disease

As Dent disease is a genetic disease caused by deleterious mutations to genes including CLCN5. As such, experimental models comprising genetically modified mice are a useful tool not only for studying the biological aspects of the disease but also for developing potential treatments for Dent disease, including those of the current disclosure. Thus in another aspect, the invention includes a mouse model for studying type 1 Dent disease, wherein the CLCN5 gene in the mice is disrupted by one or more mutations. Any number of mutations may result in the inactivation or reduced activation of a particular gene by altering the structure of the resulting protein or preventing the production of a protein all together. Such mutations include but are not limited to missense, frameshift, and nonsense mutations. In certain embodiments, the mutation can be in a region that controls post-transcriptional process of the mRNA encoded in the gene including but not limited to splicing, among other processes. In certain embodiments, the mutation is a deletion that includes one or more exons of the CLCN5 gene. In certain embodiments, the deletion affects exon 3, exon 4, exon 5, exon 6, exon 7, exon 8, exon 9, exon 10, and exon 11 of the CLCN5 gene, or any combination thereof. Thus, in certain embodiments, the one or more CLCN5 mutations result in a non-functional CLCN5 protein.

In certain mouse models, the genetic mutations or alterations affect the fertility or fecundity of the animals. In many cases, these effects are deleterious, as they result in much fewer or no offspring bearing the desired genotype required by the model. One method of reducing or avoiding these negative effects on fertility and fecundity is to outbreed the experimental mice to another strain, as the severity of fertility problems are often strain-specific. As such, in certain embodiments of the current invention, the breeding of experimental animals involves the sire and dam being of different strains. In one non-limiting example, the dam is a heterozygous for the CLCN5 mutation and the sire is wildtype. This setup recapitulates the X-linked inheritance commonly seen in Dent disease. In certain embodiments, the sire is of the FVB background while the dam is of the C57BL/6 background. It is also contemplated that the sire and dam of the mouse model of the current invention could be of any number of different strains including, but not limited to BALB/C and derivatives, C3H and derivatives, DBA and derivatives, C57BL/10 and derivatives, as well as other derivatives of the C57BL/6 and FVB lines or any combination thereof. The skilled artisan would recognize the relative advantages of the various experimental mouse strains in selecting two for use in the mouse model of the current invention.

### Pharmaceutical Compositions

Pharmaceutical compositions of the present invention may comprise as described herein, in combination with one or more pharmaceutically or physiologically acceptable carriers, diluents, adjuvants or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented) and the manner or route of administration. The quantity and frequency of administration will be determined by such factors as the condition of the patient, and the type and severity of the patient's disease, although appropriate dosages may be determined by clinical trials.

Pharmaceutical compositions of the present invention may be administered in solid or liquid form such as tablets, capsules, powders, solutions, suspensions, emulsions and the like. Pharmaceutical compositions of the present invention may be administered orally, parenterally, subcutaneously, intravenously, intramuscularly, intraperitoneally, by nasal instillation, by implantation, by intracavitary or intravesical instillation, intraocularly, intraarterially, intralesionally, transdermally, or by the application to mucous membranes. In some embodiments, the composition may be applied to the nose, throat or bronchial tubes, for example by inhalation.

Optionally, the methods of the invention provide for the administration of a composition of the invention to a suitable animal model to identify the dosage of the composition(s), concentration of components therein and timing of administering the composition(s), which elicit tissue repair, reduce cell death, or induce another desirable biological response. Such determinations do not require undue experimentation, but are routine and can be ascertained without undue experimentation.

The biologically active agents can be conveniently provided to a subject as sterile liquid preparations, e.g., isotonic aqueous solutions, suspensions, emulsions, dispersions, or viscous compositions, which may be buffered to a selected pH. Lentiviral vectors and agents of the invention may be provided as liquid or viscous formulations. For some applications, liquid formations are desirable because they are convenient to administer, especially by injection. Where prolonged contact with a tissue is desired, a viscous composition may be preferred. Such compositions are formulated within the appropriate viscosity range. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions are prepared by suspending talampanel and/or perampanel in the required amount of the appropriate solvent with various amounts of the other ingredients, as desired. Such compositions may be in admixture with a suitable carrier, diluent, or excipient, such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the cells or agents present in their conditioned media.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent, such as methylcellulose. Other suitable thickening agents include, for example, xanthan gum, carboxymethyl cellulose, hydroxypropyl cellulose, carbomer, and the like. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form). Those skilled in the art will recognize that the components of the compositions should be selected to be chemically inert.

It should be understood that the method and compositions that would be useful in the present invention are not limited to the particular formulations set forth in the examples.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed herein.

The following examples are put forth so as to provide those of ordinary skill in the art with a complete disclosure and description of how to make and use the assay, screening, and therapeutic methods of the invention, and are not intended to limit the scope of what the inventors regard as their invention.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the exemplary embodiments of the present invention, and are not to be construed as limiting in any way the remainder of the disclosure.

The materials and methods used in the following experimental examples are now described.

Study Approval. Experiments were conducted in accordance with the National Research Council Publication Guide for Care and Use of Laboratory Animals, and approved by the Institutional Animal Care and Use Committee of Wake Forest University Health Sciences (Animal protocol number A19-053). Mice were kept in microisolator cages with 12-h light/dark cycles and were fed ad libitum. Carbon dioxide (CO2) overdose, which causes rapid unconsciousness followed by death, was used to euthanize mice. The mice were exposed to CO2 without being removed from their home cage, so that the animals were not stressed by handling or being moved to a new environment. The CO2 flow rate was set to displace 10% to 30% of the cage volume per minute. When the mice showed deep narcosis, they were subjected to cervical dislocation as a secondary method of euthanasia. After euthanasia, kidney tissues were collected for further analyses.

Constructs. Lentiviral vector plasmid pCSII-hCLCN5 was constructed to express codon-optimized human CLCN5 cDNA under the control of human EF1 alpha promoter. Plasmid pCSII-hCLCN5 was made by replacing the XhoI-XbaI fragment of pCSII-EF-miRFP709-hCdt (1/100) (Addgene Plasmid #80007) with a synthesized and codon optimized cDNA encoding for human CLCN5 protein (See Table 1 for cDNA and protein sequences). Gene synthesis was performed by GenScript Inc. and the sequence was confirmed by Sanger sequencing. Plasmids pMD2.G (Addgene #12259), pMDLg/pRRE (Addgene #12259) and pRSV-Rev (Addgene #12253) were purchased from Addgene and have been described previously. The ZsGreen- and GFP-expressing lentiviral transfer plasmids pLVX-IRES-ZsGreen1 and CmiR0001-MR03 were purchased from Takara Bio and GeneCopoeia, Inc. respectively. Sequence information for primers are listed in Supplementary Table 1.

Generation of CLCN5 null mice. CLCN5 null mutant mice were generated by CRISPR/Cas9 mediated knockout of mouse Clcn5 gene. Three single guide RNAs (sgRNA), targeting mouse Clcn5 intron 2 (gRNA1: UCUGGGUUGAUCAUCUAAAC (SEQ ID NO: 14)), intron 5 (gRNA2: AGGGGGCCGAAUUCUUGCAA (SEQ ID NO: 15)) and exon 12 (gRNA3: GCAAUGCUAACUAGUAGACG (SEQ ID NO: 16)) respectively, were injected into fertilized mouse eggs with Streptococcus pyogenes Cas9 (SpCas9) mRNA to generate targeted knockout offspring. FO founder animals were identified by PCR followed by sequence analysis, which were bred to wild type mice to generate F1 animals. Successful deletion will create a strain deleting a genomic DNA region coding for 711 AA of the 746 AA CLCN5 protein (95%). RNA microinjection into fertilized eggs was done at Cyagen (Biotechnology Company, Santa Clara, California). The founder heterozygous mice in C57/BL6 background were subsequently housed in the pathogen-free animal facility at Wake Forest University Health Sciences. To avoid partial embryonic or perinatal lethality of mutant mice in C57/BL6 background, mice were bred to a 50% FVB and 50% C57/BL6 background.

Genotyping of mutant mice. Tail or ear snips were digested with proteinase K (400 μg/ml) in PCR buffer containing 0.45% NP40, at 55° C. for 3 hours or overnight. The proteinase K was inactivated at 95° C. for 13 mins. The cleared lysate was directly used for PCR. PCR primers CLCN5-KF2 (AAGGGACAGTCATGGTCTGG (SEQ ID NO: 9)) and CLCN5-KR2 (CAATGGCCTGTTGTGCATAC (SEQ ID NO: 10)) were used to amplify a product of about 1000 base pair (bp) band from the mutant allele. CLCN5-KF2 and CLCN5-W2 (CTGGGTTTCATGCATTTGTG (SEQ ID NO: 11)) were used to amplify a product of 540 bp from the wild type allele. PCR cycling included an initial denaturation at 94° C. for 5 mins, followed by 35 cycles of denaturation at 94° C. for 30 secs, annealing at 60° C. for 30 secs, and extension at 72° C. for 60 secs/kb, and a final extension step at 72° C. for 5 mins. Wild type, heterozygous and homozygous mutant mice show only the 540 bp band, both the 540 bp and the 1000 bp band, and only the 1000 bp band respectively in these two PCRs.

Isolation and culture of kidney proximal tubule cells. Kidney cortices were minced and incubated with collagenase (Worthington Biochemical, Freehold, NJ) and soybean trypsin inhibitor (GIBCO Laboratories, Grand Island, NY) both at concentrations of 0.5 mg/ml for 30 mins. After large undigested fragments were removed by gravity, the suspension was mixed with an equal volume of 10% horse serum in Hank's solution and then centrifuged at 500 revolutions/min for 7 min at room temperature. The pellets were washed once by centrifugation and then suspended in serum free cell culture medium, which was a mixture of Dulbecco's modified Eagle's medium and Ham's F-12 nutrient mixture (1:1) containing 2 mM glutamine, 15 mM N 2 hydroxyethylpiperazine-N-2-ethanesulfonic acid (HEPES), 500 U/ml penicillin, and 50 μg/ml streptomycin. The pelleted tissue pieces were resuspended in high glucose DMEM media containing 10% FBS, 1% L-glutamine and 1% penicillin streptomycin supplement, and incubated in tissue culture dishes at 37° C. 5% CO2 for the epithelia cells to grow out of the tissues and attach to the dish bottom. After two passages the cells were dissociated by trypsinization and seeded into 24 well plates at 8×104 cells/well for LV transduction.

Lentiviral vector production. Lentiviral transfer plasmid pCSII-hCLCN5, CmiR0001-MR03 and pLVX-IRES-ZsGreen1 were used to produce lentiviral vectors expressing the respective transgenes with the third generation packaging system. Briefly, 13 million actively proliferating HEK293T cells in 15-cm dish were changed into 15 ml Opti-MEM. The following DNA was used for co-transfection: 12 μg lentiviral transfer plasmid DNA (pCSII-hCLCN5, CmiR0001-MR03 or pLVX-IRES-ZsGreen1), 14 μg pMDLg/pRRE, 6 μg pMD2.G and 4 μg pRSV-Rev. The DNA was mixed in 1 ml Opti-MEM. In a separate tube, 108 μl polyethylenimine (1 mg/ml, PEI, Synchembio, Cat #SH-35421) was added in 1 ml Opti-MEM. The DNA mixture and the PEI mixture were then mixed and incubated at room temperature for 15 mins. The DNA/PEI mixture was then added to the cells in Opti-MEM. Twenty-four hours after transfection, the medium was changed to 15 ml Opti-MEM and the lentiviral vectors were collected 48 h and 72 h after transfection. The combined supernatants were spun for 10 min at 500 g to remove cell debris. The cleared supernatant was further processed as described below for in vivo delivery.

Concentrating lentiviral vectors. The supernatant containing lentiviral vectors was first concentrated with the KR2i TFF System (KrosFlo® Research 2i Tangential Flow Filtration System) (Spectrum Lab, Cat. No. SYR2-U20) using the concentration-diafiltration-concentration mode. Briefly, 150-300 ml supernatant was first concentrated to about 50 ml, diafiltrated with 1000 ml PBS, and finally concentrated to about 8 ml. The hollow fiber filter modules were made from modified polyethersulfone, with a molecular weight cut-off of 500 kDa. The flow rate and the pressure limit were 80 ml/min and 8 psi for the filter module D02-E500-05-N, and 10 ml/min and 5 psi for the filter module CO2-E500-05-N.

To further increase the vector concentration for in vivo delivery, four volumes of TFF concentrated vectors were laid on one volume of 10% sucrose buffer (in 50 mM Tris-HCl, pH 7.4, 100 mM NaCl, 0.5 mM EDTA). The viral vectors were centrifuged at 10000 g 4° C. for 4 hours and re-suspended in −0.5 ml PBS. The vectors were aliquoted into 100 μl/tube and frozen at −80° C. for future use.

Lentiviral vector quantification. Concentrations of lentiviral vectors were determined by p24 (a capsid antigen) based ELISA (Cell Biolabs, QuickTiter™ Lentivirus Titer Kit Catalog Number VPK-107). Concentrated vectors were diluted for 200 fold for assay. To assay un-concentrated samples, the viral particles were precipitated according to the manufacturer's instructions so that the soluble p24 protein was not detected.

Retrograde ureteral injection. Lentiviral vectors were delivered to the kidney by retrograde ureteral injection as previously reported. Mice were anesthetized with 3% isoflurane inhalation and the left kidney was exposed via a 2-cm flank incision and gently separated from the surrounding fat. An atraumatic vascular clip (S&T Vascular Clamps Cat #00400-03, Fine Science Tool, Heidelberg, Germany) was placed on the ureter below the injection site to prevent leakage to the bladder. Using a 30-gauge ½ needle connected to lml syringe, lentiviral particles were injected into the ureter just below the ureteropelvic junction. The total volume of viral solution did not exceed 100 μl. The concentration of the viral vectors was 2-4 ng/μl. After 5-15 min the clamp was removed and the surgical site was closed in two layers with absorbable 5-0 Vicryl suture. If bilateral injections were performed, the same procedure was repeated on the right kidney after the closure of the left incision. Right after the surgery and before wake up, 5-10 mg/kg carprofen were be provided for three doses (one per 24 hrs). Together with the first carprofen injection, buprenorphine SR (0.5-1.0 mg/kg) was also provided via subcutaneous injection. The mice were singly housed after waking up from the surgery. Single housing was found to prevent wound damage by cage mates.

Urine collection. Mice were housed in Hatteras Instruments Model MMC100 Metabolic Mouse Cage (Hatteras Instruments Inc, 105 Southbank Dr, Cary, North Carolina) for 24 hours for urine collection. The urine samples were briefly spun at 1000 g for 5 minutes to remove possible particles. Urine volume was measured by 200 μl pipette.

Urine biochemistry. Urine calcium concentration was determined with the Calcium Assay Kit (Colorimetric) (ab102505, AbCam). Urine samples from wild type and CLCN5 LV treated mice were diluted 3.6 times and those from untreated mutant mice were diluted 10 times with water before assay. The total calcium excretion was calculated by multiplying the calcium concentration by the respective urine volume collected during 24 hours. Urine total protein concentration was determined by the Pierce™ BCA Protein Assay kit (Cat #23225). All urine samples were diluted 10 times with water before assay. The total urine protein excretion was calculated by multiplying the urine protein concentration by the respective urine volume collected during 24 hours. Urine creatinine was assayed with the Mouse Creatinine Assay Kit (Crystal Chem Inc., #80350). Urine samples were diluted 10 times with saline before assay. All measurements were performed according to the instructions of the kits.

SDS-PAGE and Western blotting analyses. Mouse kidney tissues were lysed in RIPA buffer with protease inhibitors (0.5 mM PMSF and 1× Complete Protease Inhibitor Cocktail, Roche Diagnostics Corporation, Indianapolis, IN, USA), and phosphatase inhibitors (50 mM NaF, 1.5 mM Na3VO3), and the lysates were mixed with Laemmli buffer for SDS-PAGE for Western blotting analyses. Cultured cells and urine samples were lysed directly in 1× Laemmli buffer containing protease inhibitors and phosphatase inhibitors. Anti-(3-actin antibody was from Sigma (A5441, 1:5000; St Louis, MO, USA), CLCN5 Rabbit polyclonal antibody from GeneTex (GTX53963, 1:500, Irvine, CA, USA), CC16 Rabbit polyclonal antibody from BioVendor (RD181022220-01, 1:500, Asheville, NC, USA), albumin Goat polyclonal antibody from Bethyl Laboratories (A80-129A, 1:1000, TX, USA), DBP Rabbit polyclonal antibody from Proteintech (16922-1-AP, 1:1000, IL, USA) and megalin rabbit polyclonal antibody (ab76969, AbCam) from AbCam. HRP conjugated anti-Mouse IgG (H+L) (ThermoFisher Scientific, Cat No. 31430, 1:5000) and anti-Rabbit IgG (H+L) (Cat No. 31460, 1:5000) secondary antibodies were used in Western blotting. Chemiluminescent reagents (ThermoFisher) were used to visualize the protein signals under the LAS-3000 system (Fujifilm).

Immunofluorescent analysis. Kidney tissues were fixed in 4% paraformaldehyde/PBS at 4° C. overnight. Some of the tissues were embedded in OCT for cryosections, and some were dehydrated and embedded in paraffin. Paraffin sections of 5-8 μm were prepared for histological and immunofluorescent analyses. For immunofluorescent staining, the deparaffined and rehydrated sections were incubated with primary antibodies (1:200 for CLCN5 and megalin antibodies) following blocking, and were then incubated in Alexa fluor 488 or CF-594 conjugated secondary antibodies. Sections were mounted in mounting medium with DAPI (Vector Laboratories). Images were acquired with an Axio M1 microscope equipped with an AxioCam MRc digital camera (Carl Zeiss, Thornwood, NY, USA). Different images were assembled into one file with Adobe Photoshop, with necessary resizing, rotation, and cropping. Fluorescent intensity was analyzed by NIH ImageJ (1.49v)

Vector DNA detection. Each kidney was cut into 12 pieces and one of the pieces was used for genomic DNA isolation using the DNeasy Blood & Tissue Kit (Qiagen). To detect lentiviral vector integration, the Psi sequence from the lentiviral vector was detected by qPCR, using Psi-F and Psi-R primers and SYBR Green Master Mix (Thermo Fisher Scientific). Mouse Gapdh was used as internal control, with TaqMan Universal PCR Master Mix and Gapdh Taqman probe (Thermo Fisher Scientific) used for qPCR detection.

RNA isolation and RT-qPCR analyses A miRNeasy Mini Kit (QIAGEN Cat No. 217004) was used to isolate total RNA from tissues and cultured cells. The QuantiTect Reverse Transcription Kit (QIAGEN) was used to reverse-transcribe the RNA to cDNA. RT-qPCR was run on a QuantStudio3™ or ABI 7500 instrument with primers listed in Supplementary Table 1.

Statistical Analysis. Statistical assessments were performed on urine parameter and immunostaining data using GraphPad Prism (V5) software. Data are presented as mean±standard error of the mean (SEM). For data analyses involving two groups, statistical differences between groups were calculated using two tailed t-tests. For those involving more than two groups, one-way analysis of variance (ANOVA) was performed for all parameters. When an ANOVA revealed significance, Tukey's posttests were performed for data analysis. For those involving more than one factors, two-way analysis of variance (ANOVA) was performed for all parameters. When an ANOVA revealed significance, Bonferroni posttests were performed for data analysis. Significance was set at *p<0.05, **p<0.01 and ***p<0.0001.

TABLE 1
Sequences of vectors, polynucleotides, proteins, and primers used in the
invention

| SEQ | | |
|---|---|---|
| ID | | |
| NO: | Name: | Sequence: |
| 1. | hCLCN5 | gacggatcgggagatctcccgatcccctatggtcgactctcagtacaatctgctctgatgccgcat |
| | lentiviral | agttaagccagtatctgctccctgcttgtgtgttggaggtcgctgagtagtgcgcgagcaaaattta |
| | vector | agctacaacaaggcaaggcttgaccgacaattgcatgaagaatctgcttagggttaggcgttttgc |
| | | gctgcttcgcgatgtacgggccagatatacgcgttgacattgattattgactagttattaatagtaatc |
| | | aattacggggtcattagttcatagcccatatatggagttccgcgttacataacttacggtaaatggcc |
| | | cgcctggctgaccgcccaacgacccccgcccattgacgtcaataatgacgtatgttoccatagta |
| | | acgccaatagggactttccattgacgtcaatgggtggagtatttacggtaaactgcccacttggca |
| | | gtacatcaagtgtatcatatgccaagtacgccccctattgacgtcaatgacggtaaatggcccgcc |
| | | tggcattatgcccagtacatgaccttatgggactttcctacttggcagtacatctacgtattagtcatc |
| | | gctattaccatggtgatgcggttttggcagtacatcaatgggcgtggatagcggtttgactcacgg |
| | | ggatttccaagtctccaccccattgacgtcaatgggagtttgttttggcaccaaaatcaacgggact |
| | | ttccaaaatgtcgtaacaactccgccccattgacgcaaatgggcggtaggcgtgtacggtggga |
| | | ggtctatataagcagcgcgttttgcctgtactgggtctctctggttagaccagatctgagcctggga |
| | | gctctctggctaactagggaacccactgcttaagcctcaataaagcttgccttgagtgcttcaagta |
| | | gtgtgtgcccgtctgttgtgtgactctggtaactagagatccctcagacccttttagtcagtgtggaa |
| | | aatctctagcagtggcgcccgaacagggacttgaaagcgaaagggaaaccagaggagctctct |
| | | cgacgcaggactcggcttgctgaagcgcgcacggcaagaggcgaggggcggcgactggtga |
| | | gtacgccaaaaattttgactagcggaggctagaaggagagagatgggtgcgagagcgtcagtat |
| | | taagcgggggagaattagatcgcgatgggaaaaaattcggttaaggccagggggaaagaaaaa |
| | | atataaattaaaacatatagtatgggcaagcagggagctagaacgattcgcagttaatcctggcct |
| | | gttagaaacatcagaaggctgtagacaaatactgggacagctacaaccatcccttcagacaggat |
| | | cagaagaacttagatcattatataatacagtagcaaccctctattgtgtgcatcaaaggatagagat |
| | | aaaagacaccaaggaagctttagacaagatagaggaagagcaaaaaaaagtaagaccaccg |
| | | cacagcaagcggccggccgctgatcttcagacctggaggaggagatatgagggacaattggag |
| | | aagtgaattatataaatataaagtagtaaaaattgaaccattaggagtagcacccaccaaggcaaa |
| | | gagaagagtggtgcagagagaaaaaagagcagtgggaataggagctttgttccttgggttcttgg |
| | | gagcagcaggaagcactatgggcgcagcgtcaatgacgctgacggtacaggccagacaattat |
| | | tgtctggtatagtgcagcagcagaacaatttgctgagggctattgaggcgcaacagcatctgttgc |
| | | aactcacagtctggggcatcaagcagctccaggcaagaatcctggctgtggaaagatacctaaa |
| | | ggatcaacagctcctggggatttggggttgctctggaaaactcatttgcaccactgctgtgccttgg |
| | | aatgctagttggagtaataaatctctggaacagatttggaatcacacgacctggatggagtgggac |
| | | agagaaattaacaattacacaagcttaatacactccttaattgaagaatcgcaaaaccagcaagaa |
| | | aagaatgaacaagaattattggaattagataaatgggcaagtttgtggaattggtttaacataacaa |
| | | attggctgtggtatataaaattattcataatgatagtaggaggcttggtaggtttaagaatagtttttgc |
| | | tgtactttctatagtgaatagagttaggcagggatattcaccattatcgtttcagacccacctcccaa |
| | | ccccgaggggacccgacaggcccgaaggaatagaagaagaaggtggagagagagacagag |
| | | acagatccattcgattagtgaacggatctacaaatggcagtattcatccacaattttaaaagaaaag |
| | | gggggattggggggtacagtgcaggggaaagaatagtagacataatagcaacagacatacaaa |
| | | ctaaagaattacaaaaacaaattacaaaaattcaaaattttcgggtttattacagggacagcagaaa |
| | | ttcactttgaattaattcaagcttcgtgaggctccggtgcccgtcagtgggcagagcgcacatcgc |
| | | ccacagtccccgagaagttggggggaggggtcggcaattgaaccggtgcctagagaaggtgg |
| | | cgcggggtaaactgggaaagtgatgtcgtgtactggctccgcctttttcccgagggtgggggag |
| | | aaccgtatataagtgcagtagtcgccgtgaacgttctttttcgcaacgggtttgccgccagaacac |
| | | aggtaagtgccgtgtgtggttcccgcgggcctggcctctttacgggttatggcccttgcgtgccttg |
| | | aattacttccacctggctccagtacgtgattcttgatcccgagctggagccaggggcgggccttgc |
| | | gctttaggagccccttcgcctcgtgcttgagttgaggcctggcctgggcgctggggccgccgcgt |
| | | gcgaatctggtggcaccttcgcgcctgtctcgctgctttcgataagtctctagccatttaaaatttttg |
| | | atgacctgctgcgacgctttttttctggcaagatagtcttgtaaatgcgggccaggatctgcacact |
| | | ggtatttcggtttttggggccgcgggcggcgacggggcccgtgcgtcccagcgcacatgttcgg |
| | | cgaggcggggcctgcgagcgcggccaccgagaatcggacgggggtagtctcaagctggccg |
| | | gcctgctctggtgcctggcctcgcgccgccgtgtatcgccccgccctgggcggcaaggctggc |
| | | ccggtcggcaccagttgcgtgagcggaaagatggccgcttcccggccctgctccagggggctc |
| | | aaaatggaggacgcggcgctcgggagagcggggggtgagtcacccacacaaaggaaagg |
| | | ggcctttccgtcctcagccgtcgcttcatgtgactccacggagtaccgggcgccgtccaggcacc |
| | | tcgattagttctggagcttttggagtacgtcgtctttaggttggggggaggggttttatgcgatggag |
| | | tttccccacactgagtgggtggagactgaagttaggccagcttggcacttgatgtaattctccttgg |
| | | aatttgccctttttgagtttggatcttggttcattctcaagcctcagacagtggttcaaagtttttttcttc |
| | | catttcaggtgtcgtgaacacgctaccggtctcgagccaccATGGATTTCCTgGAG |
| | | GAACCAATACCAGGTGTAGGAACATATGACGATTTCAATA |
| | | CTATAGACTGGGTGCGAGAGAAATCACGCGATCGAGACAG |
| | | ACACCGGGAGATCACGAATAAGTCTAAGGAATCTACCTGG |
| | | GCCCTCATTCACAGTGTGTCAGACGCTTTTAGCGGATGGCT |
| | | GCTTATGCTTCTGATTGGACTTCTTAGTGGTAGTTTGGCGG |
| | | GCCTGATAGACATTAGCGCGCACTGGATGACTGATCTTAA |
| | | AGAAGGCATATGCACGGGGGGATTTTGGTTCAACCACGAA |
| | | CATTGCTGCTGGAACTCCGAGCATGTGACATTCGAGGAGA |
| | | GGGACAAGTGCCCCGAGTGGAATAGTTGGAGCCAACTGAT |
| | | AATTTCTACAGATGAGGGGGCTTTTGCCTATATAGTTAATT |
| | | ATTTCATGTATGTTTTGTGGGCCCTCCTCTTCGCCTTCCTCG |
| | | CGGTATCCCTCGTTAAGGTCTTTGCCCCATATGCCTGTGGC |
| | | TCTGGTATTCCAGAAATAAAAACTATCCTTTCTGGATTTAT |
| | | AATCAGGGGATATCTGGGCAAGTGGACGTTGGTCATTAAG |
| | | ACAATCACCCTTGTCCTTGCTGTATCTTCAGGGTTGTCCTT |
| | | GGGCAAAGAGGGTCCTCTCGTTCACGTAGCTTGCTGCTGT |
| | | GGGAACATCCTTTGCCATTGTTTCAATAAATATAGGAAGA |
| | | ACGAAGCAAAGCGCCGAGAAGTTCTGAGCGCAGCAGCGG |
| | | CCGCAGGTGTCAGTGTTGCCTTCGGGGCTCCTATAGGAGG |
| | | GGTACTGTTTAGTCTCGAAGAAGTGTCATATTACTTTCCTC |
| | | TCAAGACACTGTGGAGGTCCTTTTTTGCAGCCCTGGTCGCG |
| | | GCTTTTACTCTGCGCTCTATTAATCCTTTTGGAAACAGCAG |
| | | ACTTGTGCTGTTCTACGTCGAATTCCACACCCCGTGGCATT |
| | | TGTTTGAACTCGTACCCTTTATTTTGCTGGGGATTTTCGGT |
| | | GGATTGTGGGGTGCTCTGTTCATACGCACTAACATTGCGTG |
| | | GTGCCGGAAGAGGAAGACTACTCAGTTGGGCAAATACCCA |
| | | GTTATTGAGGTCCTCGTCGTTACAGCTATCACAGCAATTCT |
| | | TGCGTTCCCCAACGAGTACACACGGATGTCTACATCCGAA |
| | | CTGATTAGCGAACTGTTCAATGATTGTGGGCTCTTGGACTC |
| | | CTCAAAACTGTGCGATTATGAAAATCGATTTAATACATCA |
| | | AAGGGCGGAGAACTTCCCGATCGGCCGGCTGGAGTGGGA |
| | | GTATACTCCGCTATGTGGCAGCTGGCGTTGACGCTCATACT |
| | | CAAAATCGTCATTACCATATTCACTTTTGGAATGAAGATTC |
| | | CCTCAGGTCTCTTTATCCCTAGTATGGCAGTTGGTGCGATT |
| | | GCGGGACGGCTCCTGGGCGTTGGCATGGAGCAGCTGGCTT |
| | | ATTACCATCAGGAGTGGACCGTATTCAATAGCTGGTGCTCT |
| | | CAGGGCGCTGATTGCATCACACCAGGCCTGTATGCCATGG |
| | | TAGGCGCTGCTGCTTGTCTTGGAGGGGTGACTAGGATGAC |
| | | GGTTTCTCTCGTCGTGATAATGTTCGAGCTTACTGGGGGTC |
| | | TTGAGTACATTGTGCCCCTGATGGCGGCGGCAATGACATC |
| | | CAAATGGGTGGCGGATGCGTTGGGTAGGGAAGGGATATAC |
| | | GATGCACATATTCGCCTTAATGGCTACCCATTTTTGGAGGC |
| | | TAAGGAAGAATTTGCACATAAAACTCTCGCCATGGATGTT |
| | | ATGAAACCGAGACGAAACGACCCATTGCTTACAGTACTTA |
| | | CACAGGATTCCATGACCGTTGAGGACGTGGAAACAATAAT |
| | | ATCTGAAACAACTTATAGTGGCTTTCCCGTCGTCGTATCCC |
| | | GAGAATCACAAAGGTTGGTAGGATTCGTGCTGCGACGCGA |
| | | CCTGATCATATCCATAGAAAACGCACGCAAGAAGCAAGAC |
| | | GGGGTAGTGTCCACGTCTATAATTTATTTCACCGAGCATAG |
| | | CCCTCCCTTGCCTCCATATACTCCGCCTACACTGAAACTTC |
| | | GAAACATCCTCGATTTGTCTCCTTTTACAGTAACCGACCTT |
| | | ACTCCAATGGAAATCGTAGTAGACATATTTAGAAAGCTTG |
| | | GATTGAGGCAATGCCTGGTTACCCACAACGGTCGGTTGCT |
| | | CGGGATAATAACGAAGAAGGACGTACTCAAACATATAGC |
| | | ACAAATGGCAAACCAGGACCCgGATTCAATCTTGTTCAACT |
| | | AGtctagagttaacatcgagggatcaagcttatcgataatcaacctctggattacaaaatttgtga |
| | | aagattgactggtattcttaactatgttgctccttttacgctatgtggatacgctgctttaatgcctttgta |
| | | tcatgctattgcttcccgtatggctttcattttctcctccttgtataaatcctggttgctgtctctttatgag |
| | | gagttgtggcccgttgtcaggcaacgtggcgtggtgtgcactgtgtttgctgacgcaacccccact |
| | | ggttggggcattgccaccacctgtcagctcctttccgggactttcgctttccccctccctattgccac |
| | | ggcggaactcatcgccgcctgccttgcccgctgctggacaggggctcggctgttgggcactgac |
| | | aattccgtggtgttgtcggggaagctgacgtcctttccatggctgctcgcctgtgttgccacctgga |
| | | ttctgcgcgggacgtccttctgctacgtcccttcggccctcaatccagcggaccttccttcccgcg |
| | | gcctgctgccggctctgcggcctcttccgcgtcttcgccttcgccctcagacgagtcggatctccc |
| | | tttgggccgcctccccgcatcgataccgtcgagacctggaaaaacatggagcaatcacaagtag |
| | | caacacagcagctaccaatgctgcttgtgcctggctagaagcacaagaggaggaggaggtggg |
| | | ttttccagtcacacctcaggtacctttaagaccaatgacttacaaggcagctgtagatcttagccact |
| | | ttttaaaagaaaaggggggactggaagggctaattcactcccaacgaagacaagatatccttgat |
| | | ctgtggatctaccacacacaaggctacttccctgattggcagaactacacaccagggccagggat |
| | | cagatatccactgacctttggatggtgctacaagctagtaccagttgagcaagagaaggtagaag |
| | | aagccaatgaaggagagaacacccgcttgttacaccctgtgagcctgcatgggatggatgaccc |
| | | ggagagagaagtattagagtggaggtttgacagccgcctagcatttcatcacatggcccgagag |
| | | ctgcatccggactgtactgggtctctctggttagaccagatctgagcctgggagctctctggctaa |
| | | ctagggaacccactgcttaagcctcaataaagcttgccttgagtgcttcaagtagtgtgtgcccgtc |
| | | tgttgtgtgactctggtaactagagatccctcagacccttttagtcagtgtggaaaatctctagcagg |
| | | gcccgtttaaacccgctgatcagcctcgactgtgccttctagttgccagccatctgttgtttgcccct |
| | | cccccgtgccttccttgaccctggaaggtgccactcccactgtcctttcctaataaaatgaggaaat |
| | | tgcatcgcattgtctgagtaggtgtcattctattctggggggtggggggggcaggacagcaagg |
| | | gggaggattgggaagacaatagcaggcatgctggggatgcggtgggctctatggcttctgagg |
| | | cggaaagaaccagctggggctctagggggtatccccacgcgccctgtagcggcgcattaagcg |
| | | cggcgggtgtggtggttacgcgcagcgtgaccgctacacttgccagcgccctagcgcccgctc |
| | | ctttcgctttcttcccttcctttctcgccacgttcgccggctttccccgtcaagctctaaatcggggcat |
| | | ccctttagggttccgatttagtgctttacggcacctcgaccccaaaaaacttgattagggtgatggtt |
| | | cacgtagtgggccatcgccctgatagacggtttttcgccctttgacgttggagtccacgttctttaat |
| | | agtggactcttgttccaaactggaacaacactcaaccctatctcggtctattcttttgatttataaggg |
| | | attttggggatttcggcctattggttaaaaaatgagctgatttaacaaaaatttaacgcgaattaattct |
| | | gtggaatgtgtgtcagttagggtgtggaaagtccccaggctccccaggcaggcagaagtatgca |
| | | aagcatgcatctcaattagtcagcaaccaggtgtggaaagtccccaggctccccagcaggcaga |
| | | agtatgcaaagcatgcatctcaattagtcagcaaccatagtcccgcccctaactccgcccatcccg |
| | | cccctaactccgcccagttccgcccattctccgccccatggctgactaattttttttatttatgcagag |
| | | gccgaggccgcctctgcctctgagctattccagaagtagtgaggaggcttttttggaggcctagg |
| | | cttttgcaaaaagctcccgggagcttgtatatccattttcggatctgatcagcacgtgttgacaattaa |
| | | tcatcggcatagtatatcggcatagtataatacgacaaggtgaggaactaaaccatggccaagttg |
| | | accagtgccgttccggtgctcaccgcgcgcgacgtcgccggagcggtcgagttctggaccgac |
| | | cggctcgggttctcccgggacttcgtggaggacgacttcgccggtgtggtccgggacgacgtga |
| | | ccctgttcatcagcgcggtccaggaccaggtggtgccggacaacaccctggcctgggtgtgggt |
| | | gcgcggcctggacgagctgtacgccgagtggtcggaggtcgtgtccacgaacttccgggacgc |
| | | ctccgggccggccatgaccgagatcggcgagcagccgtggggggggagttcgccctgcgc |
| | | gacccggccggcaactgcgtgcacttcgtggccgaggagcaggactgacacgtgctacgagat |
| | | ttcgattccaccgccgccttctatgaaaggttgggcttcggaatcgttttccgggacgccggctgg |
| | | atgatcctccagcgcggggatctcatgctggagttcttcgcccaccccaacttgtttattgcagctta |
| | | taatggttacaaataaagcaatagcatcacaaatttcacaaataaagcatttttttcactgcattctagt |
| | | tgtggtttgtccaaactcatcaatgtatcttatcatgtctgtataccgtcgacctctagctagagcttgg |
| | | cgtaatcatggtcatagctgtttcctgtgtgaaattgttatccgctcacaattccacacaacatacga |
| | | gccggaagcataaagtgtaaagcctggggtgcctaatgagtgagctaactcacattaattgcgttg |
| | | cgctcactgcccgctttccagtcgggaaacctgtcgtgccagctgcattaatgaatcggccaacg |
| | | cgcggggagaggcggtttgcgtattgggcgctcttccgcttcctcgctcactgactcgctgcgctc |
| | | ggtcgttcggctgcggcgagcggtatcagctcactcaaaggcggtaatacggttatccacagaat |
| | | caggggataacgcaggaaagaacatgtgagcaaaaggccagcaaaaggccaggaaccgtaa |
| | | aaaggccgcgttgctggcgtttttccataggctccgcccccctgacgagcatcacaaaaatcgac |
| | | gctcaagtcagaggtggcgaaacccgacaggactataaagataccaggcgtttccccctggaag |
| | | ctccctcgtgcgctctcctgttccgaccctgccgcttaccggatacctgtccgcctttctcccttcgg |
| | | gaagcgtggcgctttctcaatgctcacgctgtaggtatctcagttcggtgtaggtcgttcgctccaa |
| | | gctgggctgtgtgcacgaaccccccgttcagcccgaccgctgcgccttatccggtaactatcgtct |
| | | tgagtccaacccggtaagacacgacttatcgccactggcagcagccactggtaacaggattagc |
| | | agagcgaggtatgtaggcggtgctacagagttcttgaagtggtggcctaactacggctacactag |
| | | aaggacagtatttggtatctgcgctctgctgaagccagttaccttcggaaaaagagttggtagctct |
| | | tgatccggcaaacaaaccaccgctggtagcggtggtttttttgtttgcaagcagcagattacgcgc |
| | | agaaaaaaaggatctcaagaagatcctttgatcttttctacggggtctgacgctcagtggaacgaa |
| | | aactcacgttaagggattttggtcatgagattatcaaaaaggatcttcacctagatccttttaaattaa |
| | | aaatgaagttttaaatcaatctaaagtatatatgagtaaacttggtctgacagttaccaatgcttaatc |
| | | agtgaggcacctatctcagcgatctgtctatttcgttcatccatagttgcctgactccccgtcgtgta |
| | | gataactacgatacgggagggcttaccatctggccccagtgctgcaatgataccgcgagaccca |
| | | cgctcaccggctccagatttatcagcaataaaccagccagccggaagggccgagcgcagaagt |
| | | ggtcctgcaactttatccgcctccatccagtctattaattgttgccgggaagctagagtaagtagttc |
| | | gccagttaatagtttgcgcaacgttgttgccattgctacaggcatcgtggtgtcacgctcgtcgtttg |
| | | gtatggcttcattcagctccggttcccaacgatcaaggcgagttacatgatcccccatgttgtgcaa |
| | | aaaagcggttagctccttcggtcctccgatcgttgtcagaagtaagttggccgcagtgttatcactc |
| | | atggttatggcagcactgcataattctcttactgtcatgccatccgtaagatgcttttctgtgactggt |
| | | gagtactcaaccaagtcattctgagaatagtgtatgcggcgaccgagttgctcttgcccggcgtca |
| | | atacgggataataccgcgccacatagcagaactttaaaagtgctcatcattggaaaacgttcttcg |
| | | gggcgaaaactctcaaggatcttaccgctgttgagatccagttcgatgtaacccactcgtgcaccc |
| | | aactgatcttcagcatcttttactttcaccagcgtttctgggtgagcaaaaacaggaaggcaaaatg |
| | | ccgcaaaaaagggaataagggcgacacggaaatgttgaatactcatactcttcctttttcaatattat |
| | | tgaagcatttatcagggttattgtctcatgagcggatacatatttgaatgtatttagaaaaataaacaa |
| | | ataggggttccgcgcacatttccccgaaaagtgccacctgacgtc |
| 2 | hCLCN5 | ATGGATTTCCTgGAGGAACCAATACCAGGTGTAGGAACAT |
| | CDNA | ATGACGATTTCAATACTATAGACTGGGTGCGAGAGAAATC |
| | | ACGCGATCGAGACAGACACCGGGAGATCACGAATAAGTCT |
| | | AAGGAATCTACCTGGGCCCTCATTCACAGTGTGTCAGACG |
| | | CTTTTAGCGGATGGCTGCTTATGCTTCTGATTGGACTTCTT |
| | | AGTGGTAGTTTGGCGGGCCTGATAGACATTAGCGCGCACT |
| | | GGATGACTGATCTTAAAGAAGGCATATGCACGGGGGGATT |
| | | TTGGTTCAACCACGAACATTGCTGCTGGAACTCCGAGCAT |
| | | GTGACATTCGAGGAGAGGGACAAGTGCCCCGAGTGGAAT |
| | | AGTTGGAGCCAACTGATAATTTCTACAGATGAGGGGGCTT |
| | | TTGCCTATATAGTTAATTATTTCATGTATGTTTTGTGGGCCC |
| | | TCCTCTTCGCCTTCCTCGCGGTATCCCTCGTTAAGGTCTTTG |
| | | CCCCATATGCCTGTGGCTCTGGTATTCCAGAAATAAAAACT |
| | | ATCCTTTCTGGATTTATAATCAGGGGATATCTGGGCAAGTG |
| | | GACGTTGGTCATTAAGACAATCACCCTTGTCCTTGCTGTAT |
| | | CTTCAGGGTTGTCCTTGGGCAAAGAGGGTCCTCTCGTTCAC |
| | | GTAGCTTGCTGCTGTGGGAACATCCTTTGCCATTGTTTCAA |
| | | TAAATATAGGAAGAACGAAGCAAAGCGCCGAGAAGTTCT |
| | | GAGCGCAGCAGCGGCCGCAGGTGTCAGTGTTGCCTTCGGG |
| | | GCTCCTATAGGAGGGGTACTGTTTAGTCTCGAAGAAGTGT |
| | | CATATTACTTTCCTCTCAAGACACTGTGGAGGTCCTTTTTT |
| | | GCAGCCCTGGTCGCGGCTTTTACTCTGCGCTCTATTAATCC |
| | | TTTTGGAAACAGCAGACTTGTGCTGTTCTACGTCGAATTCC |
| | | ACACCCCGTGGCATTTGTTTGAACTCGTACCCTTTATTTTG |
| | | CTGGGGATTTTCGGTGGATTGTGGGGTGCTCTGTTCATACG |
| | | CACTAACATTGCGTGGTGCCGGAAGAGGAAGACTACTCAG |
| | | TTGGGCAAATACCCAGTTATTGAGGTCCTCGTCGTTACAGC |
| | | TATCACAGCAATTCTTGCGTTCCCCAACGAGTACACACGG |
| | | ATGTCTACATCCGAACTGATTAGCGAACTGTTCAATGATTG |
| | | TGGGCTCTTGGACTCCTCAAAACTGTGCGATTATGAAAATC |
| | | GATTTAATACATCAAAGGGCGGAGAACTTCCCGATCGGCC |
| | | GGCTGGAGTGGGAGTATACTCCGCTATGTGGCAGCTGGCG |
| | | TTGACGCTCATACTCAAAATCGTCATTACCATATTCACTTT |
| | | TGGAATGAAGATTCCCTCAGGTCTCTTTATCCCTAGTATGG |
| | | CAGTTGGTGCGATTGCGGGACGGCTCCTGGGCGTTGGCAT |
| | | GGAGCAGCTGGCTTATTACCATCAGGAGTGGACCGTATTC |
| | | AATAGCTGGTGCTCTCAGGGCGCTGATTGCATCACACCAG |
| | | GCCTGTATGCCATGGTAGGCGCTGCTGCTTGTCTTGGAGGG |
| | | GTGACTAGGATGACGGTTTCTCTCGTCGTGATAATGTTCGA |
| | | GCTTACTGGGGGTCTTGAGTACATTGTGCCCCTGATGGCGG |
| | | CGGCAATGACATCCAAATGGGTGGCGGATGCGTTGGGTAG |
| | | GGAAGGGATATACGATGCACATATTCGCCTTAATGGCTAC |
| | | CCATTTTTGGAGGCTAAGGAAGAATTTGCACATAAAACTC |
| | | TCGCCATGGATGTTATGAAACCGAGACGAAACGACCCATT |
| | | GCTTACAGTACTTACACAGGATTCCATGACCGTTGAGGAC |
| | | GTGGAAACAATAATATCTGAAACAACTTATAGTGGCTTTC |
| | | CCGTCGTCGTATCCCGAGAATCACAAAGGTTGGTAGGATT |
| | | CGTGCTGCGACGCGACCTGATCATATCCATAGAAAACGCA |
| | | CGCAAGAAGCAAGACGGGGTAGTGTCCACGTCTATAATTT |
| | | ATTTCACCGAGCATAGCCCTCCCTTGCCTCCATATACTCCG |
| | | CCTACACTGAAACTTCGAAACATCCTCGATTTGTCTCCTTT |
| | | TACAGTAACCGACCTTACTCCAATGGAAATCGTAGTAGAC |
| | | ATATTTAGAAAGCTTGGATTGAGGCAATGCCTGGTTACCC |
| | | ACAACGGTCGGTTGCTCGGGATAATAACGAAGAAGGACGT |
| | | ACTCAAACATATAGCACAAATGGCAAACCAGGACCCgGAT |
| | | TCAATCTTGTTCAACTAG |
| 3 | hCLCN5 | MDFLEEPIPGVGTYDDFNTIDWVREKSRDRDRHREITNKSKE |
| | protein | STWALIHSVSDAFSGWLLMLLIGLLSGSLAGLIDISAHWMTD |
| | | LKEGICTGGFWFNHEHCCWNSEHVTFEERDKCPEWNSWSQL |
| | | IISTDEGAFAYIVNYFMYVLWALLFAFLAVSLVKVFAPYACG |
| | | SGIPEIKTILSGFIIRGYLGKWTLVIKTITLVLAVSSGLSLGKEG |
| | | PLVHVACCCGNILCHCFNKYRKNEAKRREVLSAAAAAGVSV |
| | | AFGAPIGGVLFSLEEVSYYFPLKTLWRSFFAALVAAFTLRSIN |
| | | PFGNSRLVLFYVEFHTPWHLFELVPFILLGIFGGLWGALFIRTN |
| | | IAWCRKRKTTQLGKYPVIEVLVVTAITAILAFPNEYTRMSTSE |
| | | LISELFNDCGLLDSSKLCDYENRFNTSKGGELPDRPAGVGVY |
| | | SAMWQLALTLILKIVITIFTFGMKIPSGLFIPSMAVGAIAGRLL |
| | | GVGMEQLAYYHQEWTVFNSWCSQGADCITPGLYAMVGAA |
| | | ACLGGVTRMTVSLVVIMFELTGGLEYIVPLMAAAMTSKWVA |
| | | DALGREGIYDAHIRLNGYPFLEAKEEFAHKTLAMDVMKPRR |
| | | NDPLLTVLTQDSMTVEDVETIISETTYSGFPVVVSRESQRLVG |
| | | FVLRRDLIISIENARKKQDGVVSTSIIYFTEHSPPLPPYTPPTLK |
| | | LRNILDLSPFTVTDLTPMEIVVDIFRKLGLRQCLVTHNGRLLGI |
| | | ITKKDVLKHIAQMANQDPDSILFN* |
| 4 | Human | ggctccggtgcccgtcagtgggcagagcgcacatcgcccacagtccccgagaagttgggggg |
| | EF1 alpha | aggggtcggcaattgaaccggtgcctagagaaggtggcgcggggtaaactgggaaagtgatgt |
| | promoter | cgtgtactggctccgcctttttcccgaggggggggagaaccgtatataagtgcagtagtcgccgt |
| | | gaacgttctttttcgcaacgggtttgccgccagaacacaggtaagtgccgtgtgtggttcccgcgg |
| | | gcctggcctctttacgggttatggcccttgcgtgccttgaattacttccacctggctccagtacgtga |
| | | ttcttgatcccgagctggagccaggggcgggccttgcgctttaggagccccttcgcctcgtgcttg |
| | | agttgaggcctggcctgggcgctggggccgccgcgtgcgaatctggtggcaccttcgcgcctgt |
| | | ctcgctgctttcgataagtctctagccatttaaaatttttgatgacctgctgcgacgctttttttctggca |
| | | agatagtcttgtaaatgcgggccaggatctgcacactggtatttcggtttttggggccgcgggcgg |
| | | cgacggggcccgtgcgtcccagcgcacatgttcggcgaggcggggcctgcgagcgcggcca |
| | | ccgagaatcggacgggggtagtctcaagctggccggcctgctctggtgcctggcctcgcgccg |
| | | ccgtgtatcgccccgccctgggcggcaaggctggcccggtcggcaccagttgcgtgagcgga |
| | | aagatggccgcttcccggccctgctccagggggctcaaaatggaggacgcggcgctcgggag |
| | | agcgggcgggtgagtcacccacacaaaggaaaggggcctttccgtcctcagccgtcgcttcatg |
| | | tgactccacggagtaccgggcgccgtccaggcacctcgattagttctggagcttttggagtacgtc |
| | | gtctttaggttggggggaggggttttatgcgatggagtttccccacactgagtgggtggagactga |
| | | agttaggccagcttggcacttgatgtaattctccttggaatttgccctttttgagtttggatcttggttca |
| | | ttctcaagcctcagacagtggttcaaagtttttttcttccatttcaggtgtcgtga |
| 5 | hCLCN5-F | TCTCGCCATGGATGTTATGA |
| | (cDNA) | |
| | primer | |
| 6 | hCLCN5-R | TCTTGCGTGCGTTTTCTATG |
| | (cDNA) | |
| | primer | |
| 7 | mCLCN5- | CCCTGGTGTAGGGACCTATG |
| | EF primer | |
| 8 | mCLCN5- | CAGAATTCCAGCAACAGTGC |
| | ER primer | |
| 9 | CLCN5- | AAGGGACAGTCATGGTCTGG |
| | KF2 | |
| | primer | |
| 10 | CLCN5- | CAATGGCCTGTTGTGCATAC |
| | KR2 | |
| | primer | |
| 11 | CLCN5- | CTGGGTTTCATGCATTTGTG |
| | W2 | |
| | primer | |
| 12 | Psi-F | TCTCGACGCAGGACTCG |
| | primer | |
| 13 | Psi-R | TACTGACGCTCTCGCACC |
| | primer | |

### Example 1: CLCN5 Null Mice Manifest Typical Type 1 Dent Disease (DD1) Phenotypes

Dent disease is caused by the inability of kidney cells to reabsorb nutrients, water, and other materials that have been filtered from the bloodstream. High amounts of proteins and calcium in the kidney filtrate damage the kidney cells and eventually cause the observed symptoms. The studies of the current disclosure sought to develop a useful animal model for Dent's disease in order to aid in the development of a gene therapy for the disease. The ultimate goal being to correct the mutated gene in a minimal percent of patient kidney cells, so that these cells can reabsorb enough material from the kidney filtrate to prevent damage to the kidney cells and restore or maintain normal kidney function.

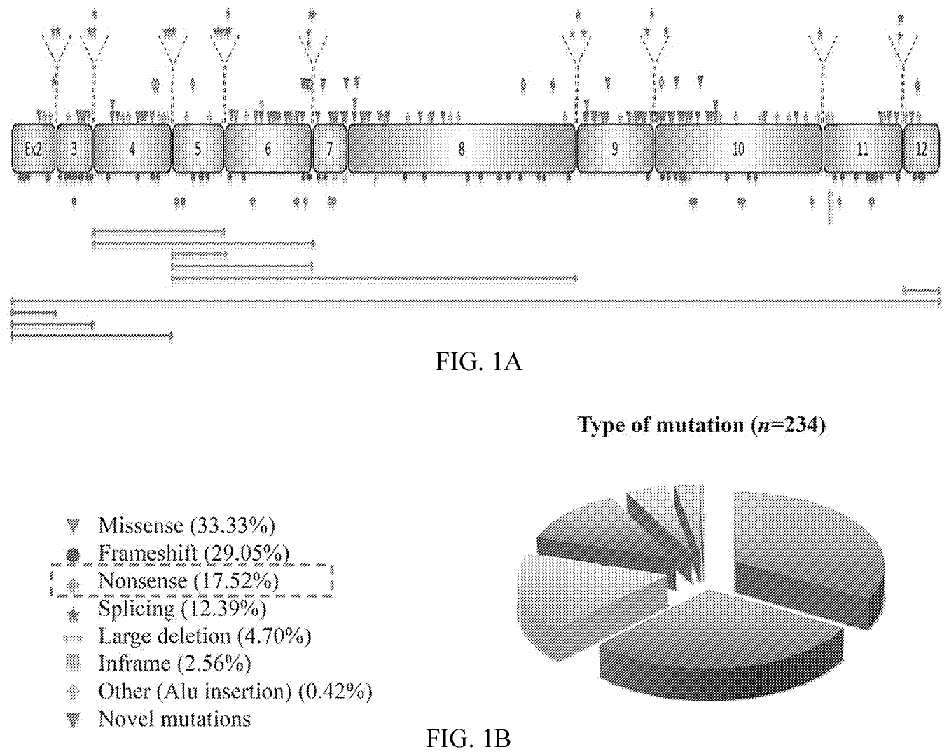

Although adverse changes in at least two genes (both X chromosome linked and inherited from the mother) can cause this disease, abnormalities in one of the genes (CLCN5 gene) are responsible for 60% of patients (type 1 Dent's disease). A wide number of mutations in CLCN5 have been identified with missense, frameshift, and nonsense mutations making up the majority (see FIG. 1). Thus in the current disclosure, a gene therapy strategy specific for type 1 Dent's disease is developed. Currently there are only supportive treatments for Dent's disease and none of them target the genetic causes of the disease.

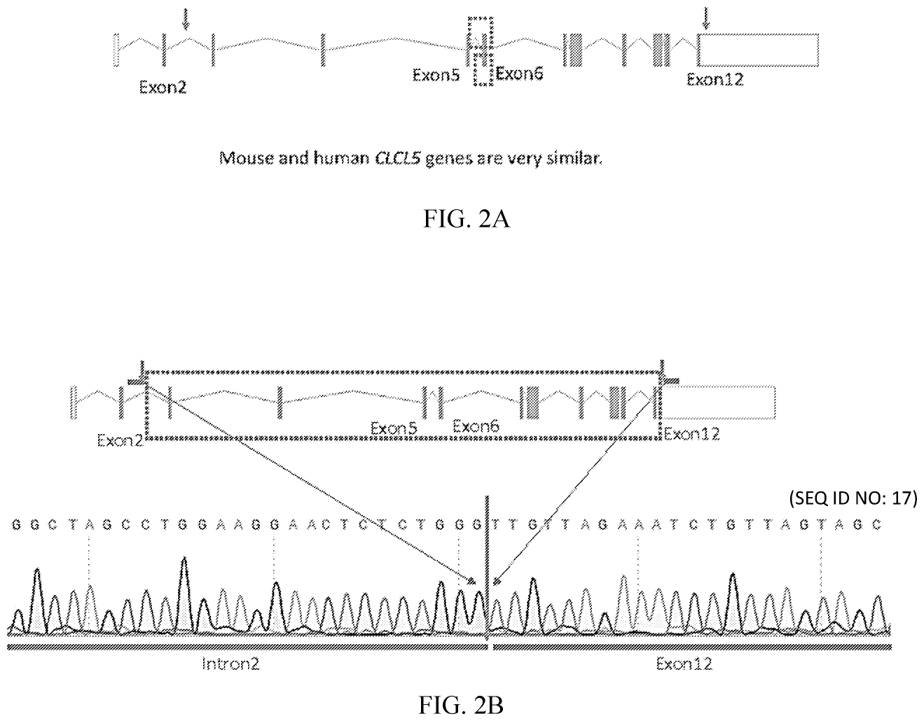

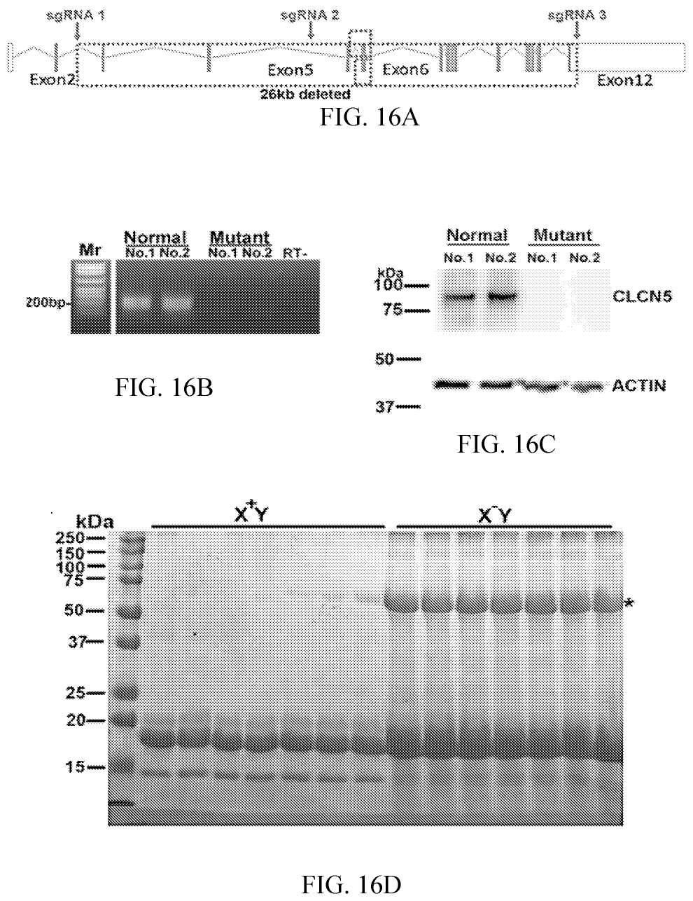

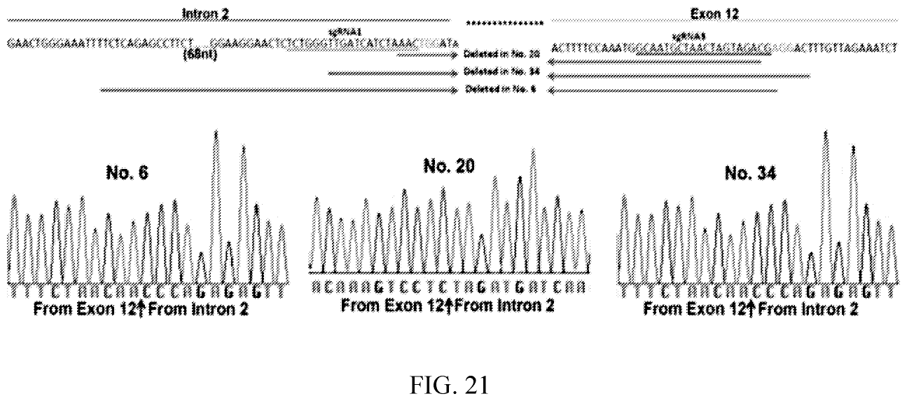

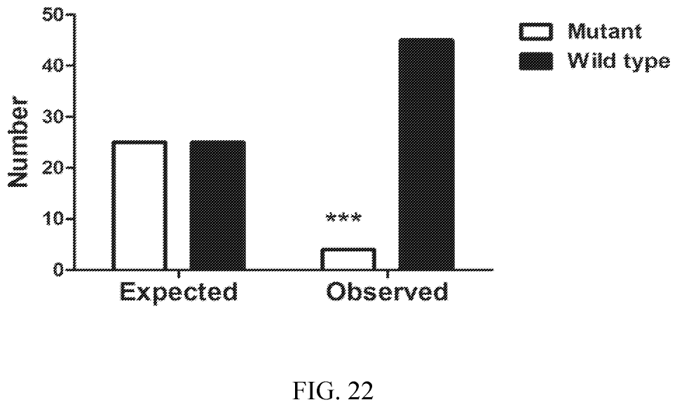

In order to develop a treatment for Dent's disease, studies were first undertaken to create a suitable mouse model of the disease. Here, CRISPR/Cas9 technology was used to target most of the coding region of the CLCN5 gene for deletion (FIG. 2A). Three guide RNAs were designed to target mouse CLCN5 gene intron 2, intron 4 and exon 12 respectively (FIG. 16A), in order to delete 95% of the protein coding region and completely disrupt the gene function. The three single guide RNAs (sgRNAs) and Cas9 mRNA were injected into fertilized mouse eggs to delete CLCN5 gene. Three heterozygous founder female mice were obtained, all had a 26 kilo bp deletion in Clcn5 gene (FIG. 21), deleting 95% of the CLCN5 coding region. There were no other known coding genes or non-coding genes within 80 kilo bps around the deleted region. Thus we postulated that the deletion of CLCN5 gene was unlikely to affect other genes. Progenies from one female carrier (No. 34) were used for subsequent studies.

Considering that the mice were generated by CRISPR/Cas9-mediated gene mutation, we analyzed possible off targets of the three sgRNAs used (three rather than two sgRNAs were used to increase the chance of deleting the whole gene). All predicted off-targets had at least 3 nt mismatch to the sgRNAs. Only one of the predicted off-targets (for sgRNA 2) hit the exon of a protein coding gene (Itgb6). DNA of this region was amplified from a male mutant mouse and sequenced. No mutation or heterozygosity was observed (FIG. 30). Eighteen predicted off-targets fell in introns and 23 in intergenic regions. The regions of all 4 predicted off-targets on the X-chromosome from a male mutant mouse were amplified, and failed to detect mutations or deletions (FIG. 30). Since the off-targets on X-chromosome link with the CLCN5 deletion and male mice have only one copy of X chromosome, successful amplification of the region also ruled out the possibility of large deletions. For off-targets on autosomal chromosomes, we sequenced the regions of all 5 off-targets with 3 nt mismatches to the sgRNAs, and 2 off-targets with 4 nt mismatches to the sgRNA (at least 3 off-targets were analyzed for each sgRNA), and failed to detect mutations or heterozygosity in any of these regions in mutant mice (FIG. 30). Altogether, the data showed that the likelihood of unintendedly mutating other genes was low.

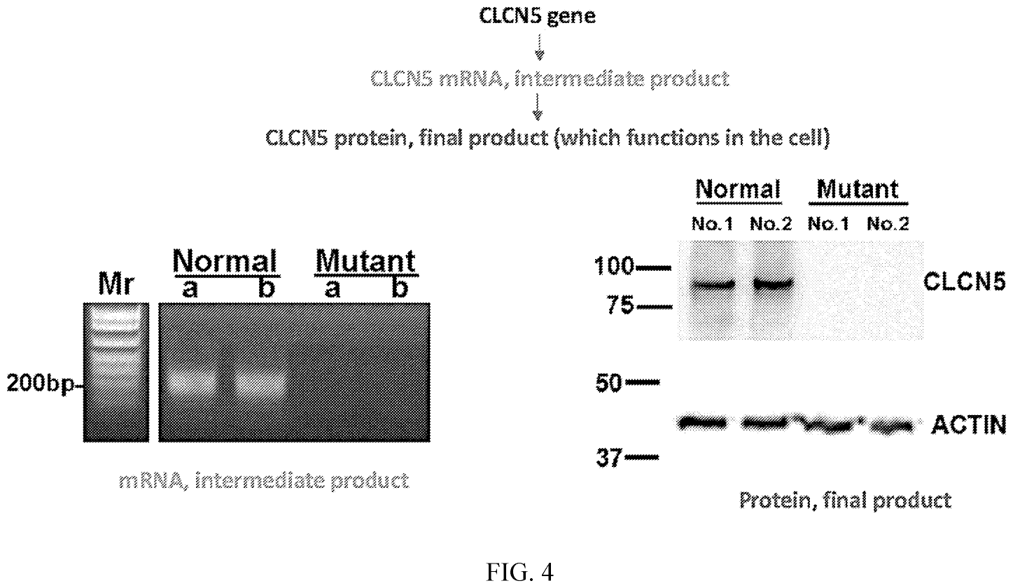

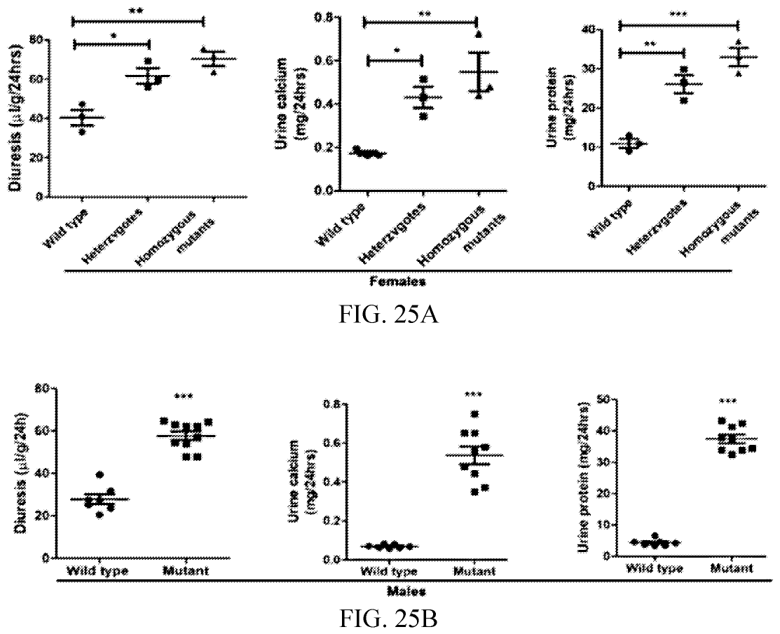

Loss of CLCN5 expression was confirmed in mutant mice by RT-PCR (FIG. 16B) and Western blotting (FIG. 16C). Mutant animals resulting from these studies were sequenced, which confirmed the excision of exons 3 through 11 (FIG. 2B), thus creating a model completely lacking CLCN5 function. Mutant mice were then confirmed to lack expression of CLCN5 gene products both by detection of CLCN5 mRNA and CLCN5 protein (FIG. 4). Urine from wild type and mutant mice was collected and measured for total urine volume, total urine protein, and calcium levels excreted during 24 hours. Female and male mutant mice showed diuresis, hypercalciuria and proteinuria (FIGS. 25A-25B). The wild type female mice showed higher urine calcium levels than wild type male mice, consistent with observation made previously. Interestingly, heterozygous female mice also showed DD1-like phenotypes that appeared less severe than homozygous mutant female mice, suggesting haploinsufficiency and consistent with reports that some human female heterozygous carriers show mild DD1 symptoms. The phenotypes observed in these null mutant mice (6-7 fold increase of urine protein and urine calcium) were much more severe than previously reported, possibly due to the deletion of majority of the Clcn5 coding sequence. Urine creatinine concentration of mutant mice was similar to that of wild type mice, suggesting that creatinine filtration in mutant mice was not greatly affected at the time of analysis.

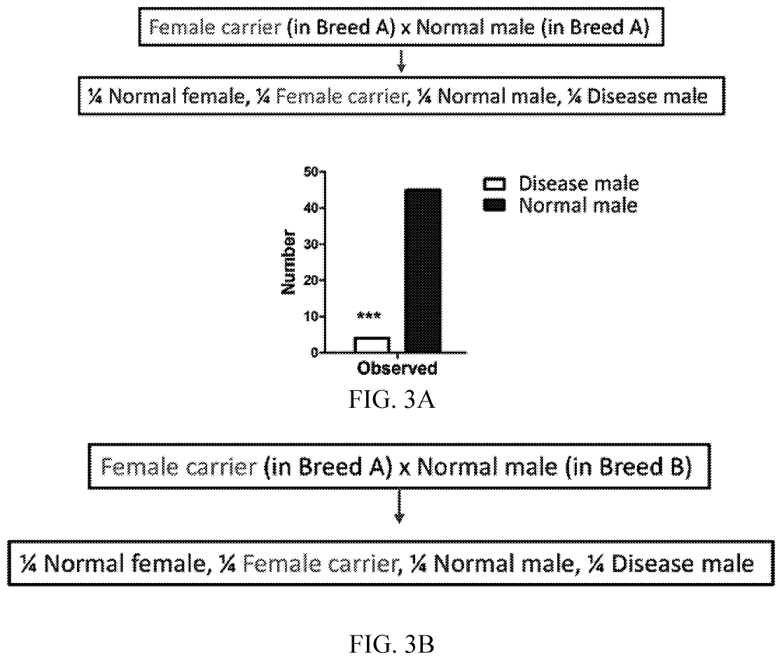

Unexpectedly, breeding difficulties resulted in obtaining insufficient numbers of diseased male mice. Ratios of observed diseased male mice to normal male mice (FIG. 3A) suggested embryonic lethality. This problem was not observed in previous studies nor in humans. To restore expected mendelian ratios of offspring, a breeding strategy was developed in which separated the female carrier and normal male into different breeds (a mix between FVB and C57BL/6) (FIG. 3B).

The mutant mice were then investigated to see if they showed phenotypes similar to those observed in DD1 patients. Urine from normal and mutant mice and measured diuresis, total urine protein, and calcium levels (Table 2). Male and female mutant mice urinated more frequently than normal mice and excreted more urine protein and calcium (Table 3). The normal female mice showed higher urine calcium levels than normal male mice, consistent with observation made previously. Interestingly, heterozygous female mice also showed DD1-like phenotypes that were less severe than homozygous mutant female mice, suggesting haploinsufficiency and consistent with report that some human female heterozygous carriers show mild DD1 symptoms. The phenotypes observed in these null mutant mice (2 fold increase of urine protein and 6 fold increase of urine calcium) were much more severe than previously reported, possibly due to the deletion of majority of the CLCN5 coding sequence. Urine creatinine concentration of mutant mice was similar to that of wild type mice, suggesting that creatinine filtration in mutant mice was not greatly affected at the time of test.

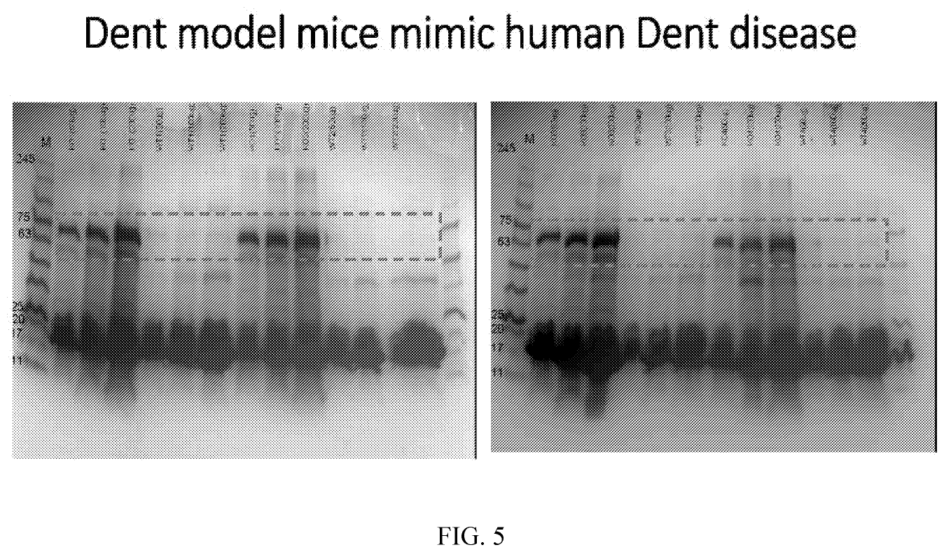

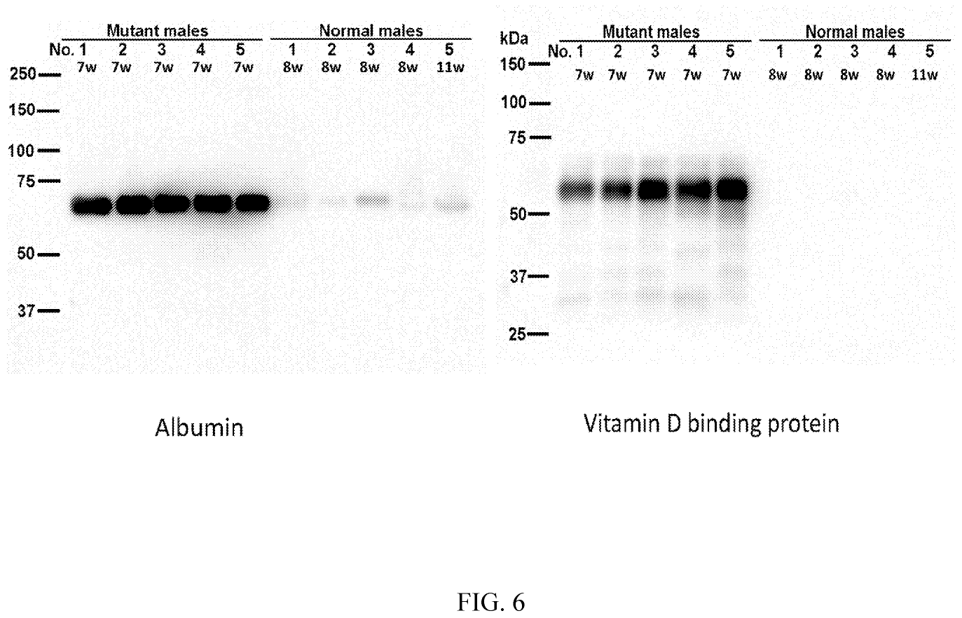

Consistent with increased total urine protein content (BCA assay) in mutant mice, SDS-PAGE analysis of urine protein confirmed increased protein content in mutant urine, with a very intense unidentified protein of 61 kDa in urine samples of mutant mice but almost invisible in those of wild type mice (FIG. 5 and FIG. 16D). The visibility of this intense band in SDS-PAGE was a reliable predictor of other DD1 phenotypes. Western blotting further confirmed the increase of urine albumin, Vitamin D binding protein (DBP) and Club cell secretory protein (CC16, also called CC10) in samples from mutant mice (FIG. 6 and FIG. 16E). We loaded equal volumes of urine samples in SDS-PAGE and Western blotting experiments. Considering the increased urine volume in mutant mice, the degree of urine protein increase was more dramatic than appeared in Western blotting analyses. The data showed that we have successfully knocked out the mouse CLCN5 gene and that the mutant mice showed more severe DD1 phenotypes than observed in published models.

TABLE 2
Urine protein and calcium values of control and mutant mice

| | Control | Mutant | P value |
|---|---|---|---|
| Age (days) | 51.4 ± 6.2 | 49.7 ± 9.0 | P = 0.87 |
| Bodyweight (g) | 25.4 ± 1.8 | 26.5 ± 2.0 | P = 0.68 |
| Urine volume | 0.037 ± 0.006 | 0.061 ± 0.004 | P = 0.0046 ** |
| (ml/g body/24 h) | | | |
| Urine protein | 3.95 ± 0.35 | 37.13 ± 2.08 | P < 0.0001*** |
| (mg/24 h) | | | |
| Urine calcium | 0.12 ± 0.01 | 0.867 ± 0.0535 | P < 0.0001*** |
| (mg/24 h) | | | |

** p < 0.01;
***p < 0.0001. Ten mice were included for each group.

TABLE 3
Urine analyses of control and mutant mice

| | Females | | | Males | |
|---|---|---|---|---|---|
| | X⁺/X⁺ | X⁺/X⁻ | X⁻/X⁻ | X⁺/Y | X⁻/Y untreated |
| | (n = 3) | (n = 3) | (n = 3) | (n = 10) | (n = 10) |
| Age (days) | 82 | 82 | 82 | 51.4 ± 6.2 | 49.7 ± 9.0 |
| Bodyweight (g) | 22.4 ± 0.86 | 20.3 ± 0.41 | 21.6 ± 0.56 | 25.4 ± 1.8 | 26.5 ± 2.0 |
| Diuresis (μl/g | 40.4 ± 4.0 | 61.7 ± 4.0* | 70.3 ± 3.5** | 36.9 ± 6.0 | 60.8 ± 4.4** |
| body/24 h) | | | | | |
| Urine protein | 10.9 ± 1.2 | 26.1 ± 2.3** | 33.0 ± 2.3*** | 4.72 ± 0.37 | 32.74 ± 2.18*** |
| (mg/24 h) | | | | | |
| Urine calcium | 0.173 ± 0.01 | 0.430 ± 0.049* | 0.548 ± 0.089** | 0.077 ± 0.006 | 0.469 ± 0.032*** |
| (mg/24 h) | | | | | |

*, **, *** indicate p < 0.05, p < 0.01 and p < 0.0001 when the indicated group was compared with wild type mice in two-tailed unpaired t-tests (males) or Tukey's Multiple Comparison Test following one-way ANOVA (females).

TABLE 4
Urine analysis of mutant mice with both kidneys treated

| | | | One kidney | | Two kidney | |
|---|---|---|---|---|---|---|
| | ZsGreen LV | | CLCN5 treated | | CLCN5 treated | |
| | treated (n = 5) | | (n = 10) | | (n = 10) | |
| | Before | After | Before | After | Before | After |
| Age (days) | 63.4 ± 19.8 | 115.2 ± 20.0 | 49.7 ± 9.0 | 79.7 ± 9.0 | 66.0 ± 16.9 | 117.6 ± 17.2 |
| Body weight | 33.4 ± 2.5 | 36.1 ± 2.1 | 26.5 ± 2.0 | 34.3 ± 1.6 | 30.9 ± 1.8 | 31.6 ± 1.6 |
| (g) | | | | | | |
| Diuresis | 51.0 ± 2.8 | 55.0 ± 0.9 | 60.9 ± 4.4 | 36.5 ± 1.8*** | 53.4 ± 2.1 | 32.9 ± 1.4*** |
| (μl/g/24 hours) | | | | | | |
| Urine protein | 40.7 ± 4.2 | 38.7 ± 3.2 | 32.8 ± 2.2 | 9.0 ± 0.5*** | 39.9 ± 1.6 | 8.3 ± 0.6*** |
| (mg/24 hours) | | | | | | |
| Urine calcium | 0.49 ± 0.08 | 0.61 ± 0.09 | 0.47 ± 0.03 | 0.06 ± 0.01*** | 0.55 ± 0.05 | 0.05 ± 0.01*** |
| (mg/24 hours) | | | | | | |

### Example 2: Development of a Gene Therapy for Type I Dent Disease

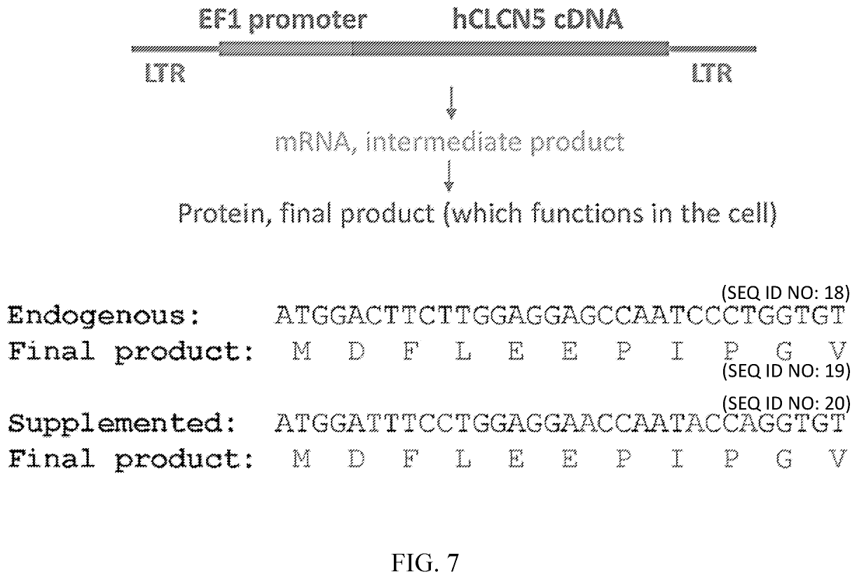

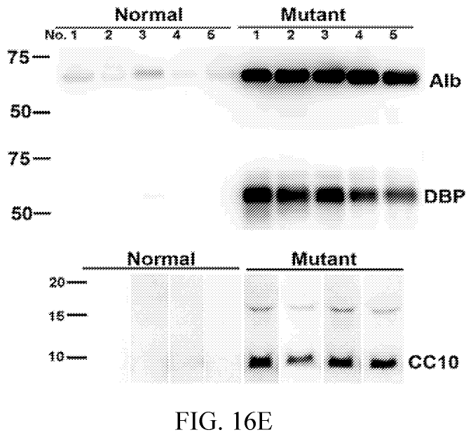

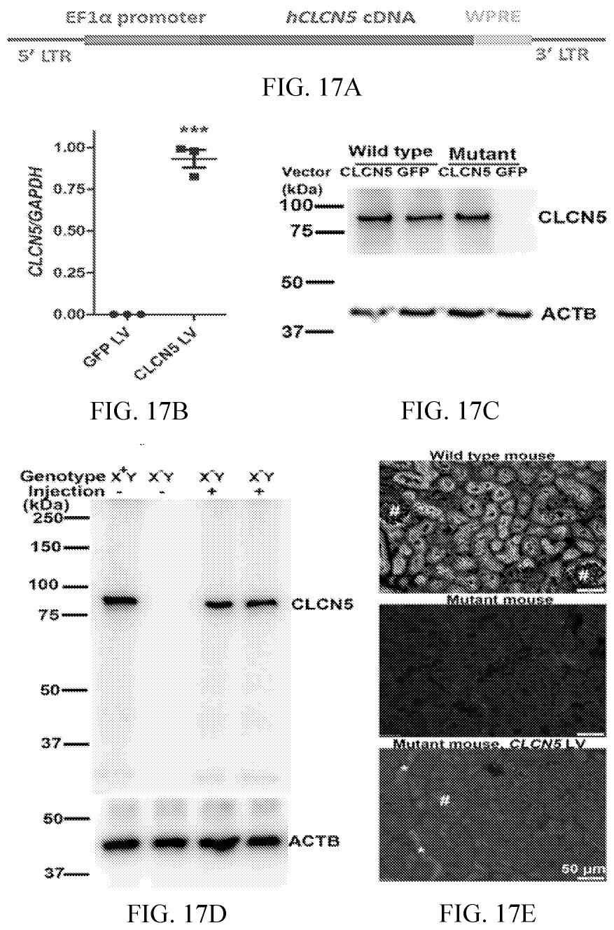

Efforts to develop a strategy to correct the mutations causing Dent disease focused on the use of a lentiviral vector to deliver a functional copy of the CLCN5 gene to cells of the kidney. Lentiviral vectors have been approved by FDA as a vehicle to deliver functional genes to human cells for gene therapy. The CLCN5-expressing lentiviral vector of the present invention was designed such that it expresses an identical final protein product but has subtle difference from the wildtype CLCN5 mRNA in sequence, so that the virus-delivered form of the mRNA can be distinguished from the original endogenous form (illustrated in FIG. 7, also see Table 1). This was accomplished by codon optimization of the transgene-expressed CLCN5 mRNA. The transfer plasmid was a third generation lentiviral expression vector containing the codon optimized human CLCN5 cDNA following the human EF1 alpha promoter (FIG. 17A, Table 1). A ubiquitously active promoter was used to test whether supplementing functional CLCN5 cDNA to the kidney is able to ameliorate the DD1 symptoms. A Woodchuck Hepatitis Virus Posttranscriptional Regulatory Element (WPRE) was included following the CLCN5 cDNA to increase target gene expression. The CLCN5-expressing lentiviral vectors were produced with a third generation packaging system and exogenous CLCN5 mRNA expression was successfully detected from HEK293T cells transduced with the lentiviral vectors (FIG. 17B).

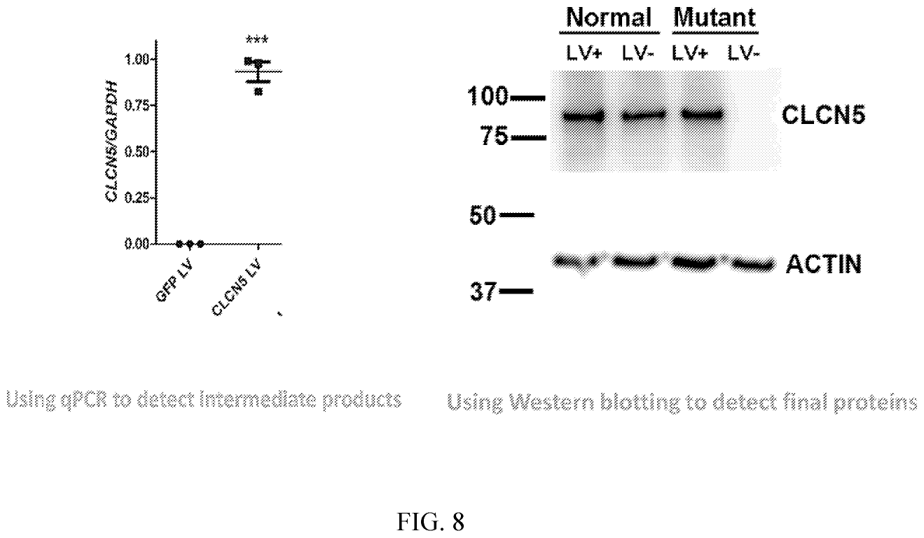

In vitro studies on human 293T cells and primary kidney cells isolated from normal and CLCN5 mutant mice transduced with the CLCN5 vector or a GFP-bearing control vector demonstrated that transduced cells expressed robust levels of CLCN5 protein (FIG. 8). Expression of the vector was then examined in isolated kidney proximal tubule cells from wild type and CLCN5 knockout mice transduced with the CLCN5 lentiviral vectors. CLCN5 protein was detected in CLCN5 LV transduced mutant cells but not in GFP-LV transduced mutant cells (FIG. 17C).

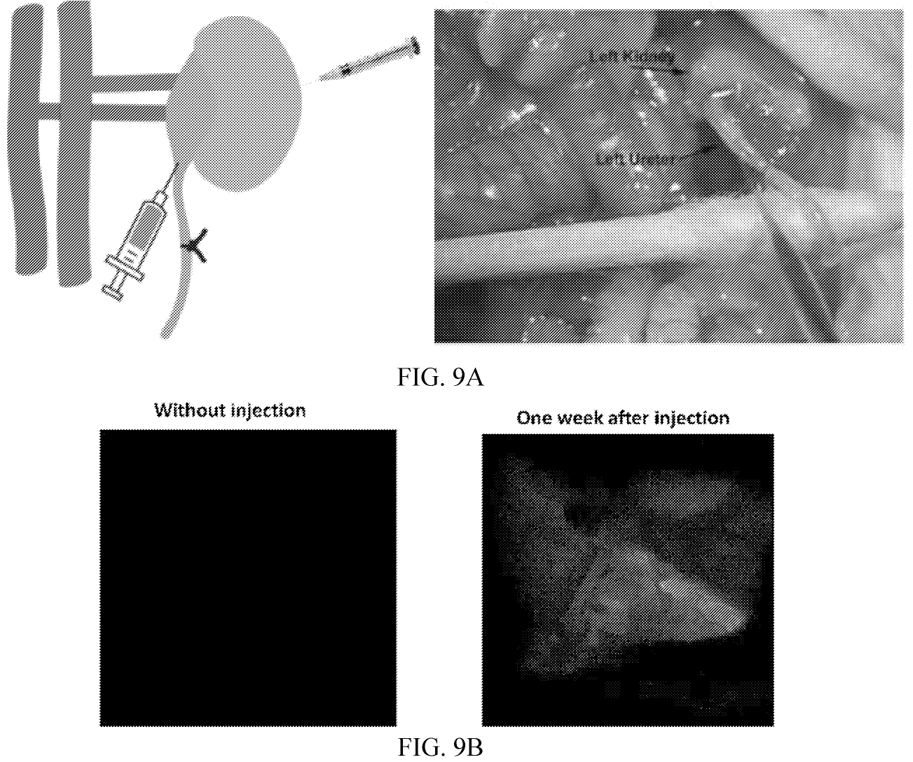

Eventual clinical use of a lentiviral CLCN5 construct will require that the viral particles are directed to the cells most in need of functional CLCN5 protein—especially those of the kidney including the proximal tubule and the thick ascending limb of Henle, both sites of calcium transport. As such, studies were undertaken delivering lentiviral vectors directly into kidney tissue by ureter ligation followed by retrograde ureteral injection. Temporarily tying-off the ureter prevents the flushing-out of lentiviral particles before they have a chance to transfect renal cells, while injection of the cells into the ureter allows viral particle access to the target tissues (FIG. 9A). In order to verify successful transduction of renal cells using this method, mice were injected with a GFP-expressing lentivirus using this technique. This method was then used to deliver 280 ng p24 of CLCN5 LV into the kidney of mutant mice. One week later, kidney tissues were harvested and assessed for GFP expression by fluorescence microscopy, which demonstrated easily visualized GFP+ cells (FIG. 9B). Western blotting analysis of protein extracted from kidney tissues found that CLCN5 protein could be detected in the injected kidneys but not from the non-injected kidneys of mutant mice (FIG. 17D). These data showed that the LV vectors could be delivered into the kidney to obtain CLCN5 expression from the delivered lentiviral vectors.

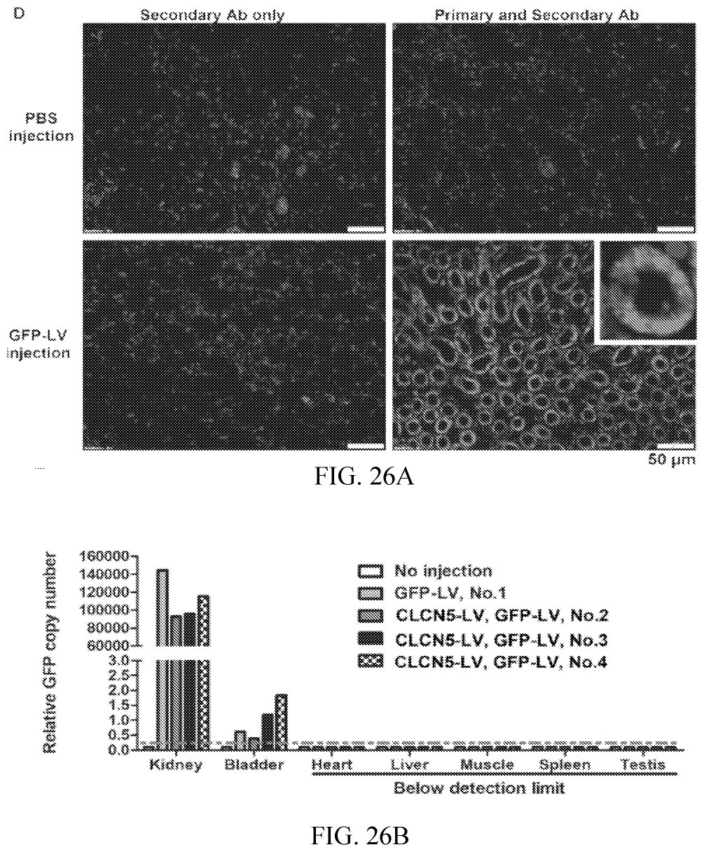

Subsequent studies tested delivering GFP LV into mouse kidney tubules using retrograde ureter injection. Two weeks following delivery of 100 μl GFP LV vectors (˜250 ng p24) to each kidney, immunofluorescence detected strong GFP expression in over 70% of the tubule structures of all four injected male mice (one 6-month wild type and three 17-month mutants) (FIG. 26A). GFP expression was not found in glomeruli, suggesting that this delivery method was more suited for delivering to tubules than to glomeruli. We collected the kidney, bladder, liver, heart, skeletal muscle, spleen and testis of the mice receiving GFP LV retrograde ureter injection, and extracted genomic DNA to detect lentiviral vector DNA. Relatively low levels of vector DNA was detected in the bladder, 10⁵ fold higher levels of vector DNA in the kidney, and undetectable vector DNA in all other organs (FIG. 26B). These data showed that retrograde ureter injection was efficient for local tubule delivery and the chance of delivering the vectors to other organs (except for the bladder and possibly other tissues of the urinary tract) was low.

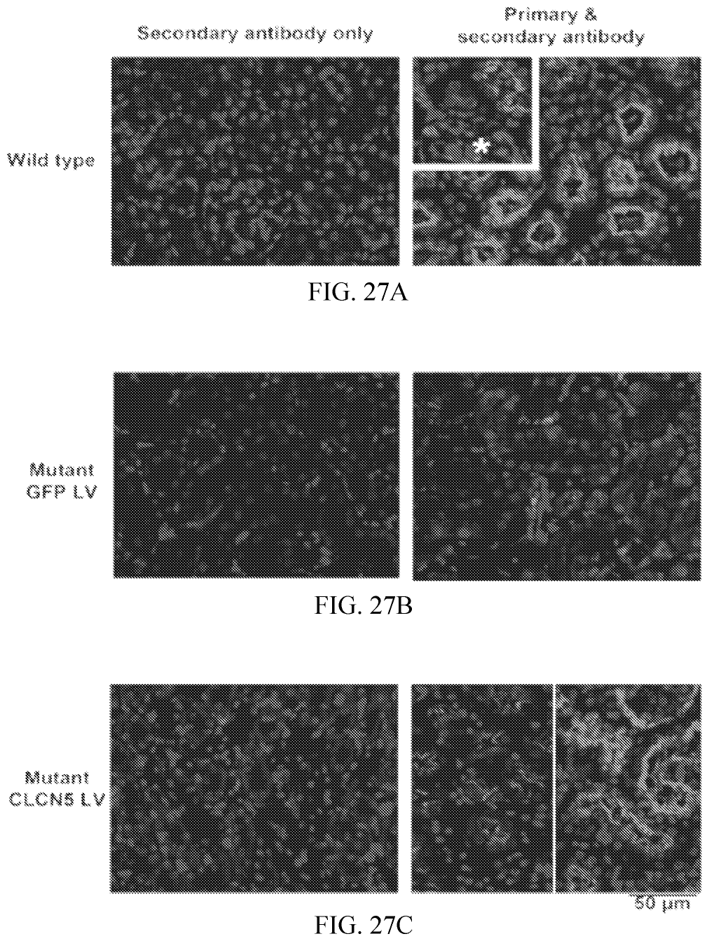

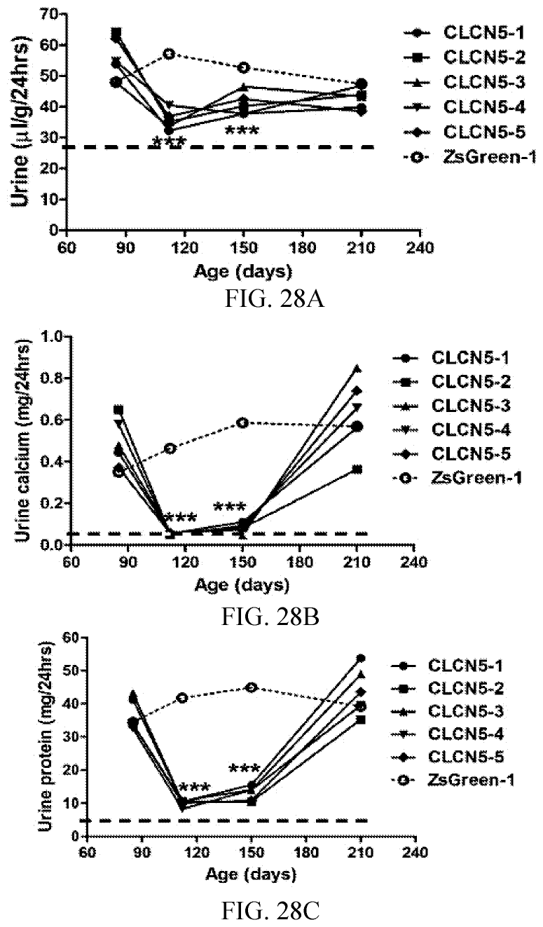

We then used this method to deliver 280 ng p24 of CLCN5 LV into the kidneys of male mutant mice. Western blotting analysis of protein extracted from kidney tissues detected CLCN5 protein in the injected kidneys but not from the non-injected kidneys two weeks after vector delivery (FIG. 17D). Immunofluorescence analysis was performed to examine the cell types expressing transgenic CLCN5. In the kidneys of wild type mice, CLCN5 was highly expressed in the proximal tubular epithelium (FIG. 27A) but weakly expressed in the glomeruli (FIG. 27A insert, marked by *). Without CLCN5 LV vector delivery, no CLCN5 expression was detected in the kidney tubules of mutant mice (FIG. 27B). Two weeks following CLCN5 LV delivery, CLCN5 was detected in kidney tubules of mutant mice (FIG. 27C). In both wild type and CLCN5 LV injected mutant mice, strongest CLCN5 signals were detected in the apical regions of the tubular cells. The apical localization of exogenous CLCN5 protein showed that the LV-expressed CLCN5 protein was corrected trafficked. The data showed that retrograde ureter injection was able to deliver the LV vectors into the kidney and result in CLCN5 expression from the delivered lentiviral vectors.

To examine the cell types expressing transgenic CLCN5, we performed immunofluorescent analysis two weeks after vector delivery. In wild type mice, CLCN5 was highly expressed in the proximal tubular epithelium (top image in FIG. 17E, marked by *) but not in the glomeruli (marked by #). Without vector delivery, no CLCN5 expression was detected in mutant mice (middle image in FIG. 17E). CLCN5 was detected in essentially all cells in the kidney of mutant animals with CLCN5 LV delivery (bottom image in FIG. 17E), including tubular structures (marked by *) and glomeruli (marked by #). Overall, the level of CLCN5 expression in LV-delivered mutant animals was lower than in wild type mice. CLCN5 expression via LV vectors was consistent with the ubiquitous EF1 alpha promoter used to control CLCN5 expression.

### Example 3: Delivering Human CLCN5 Lentiviral Vector to the Kidneys of Mutant Mice Ameliorated DD1 Phenotypes

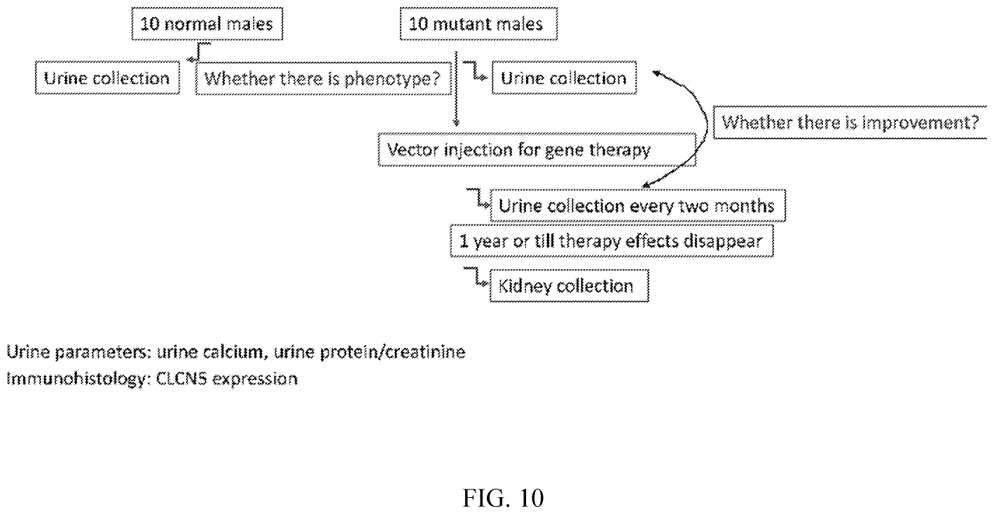

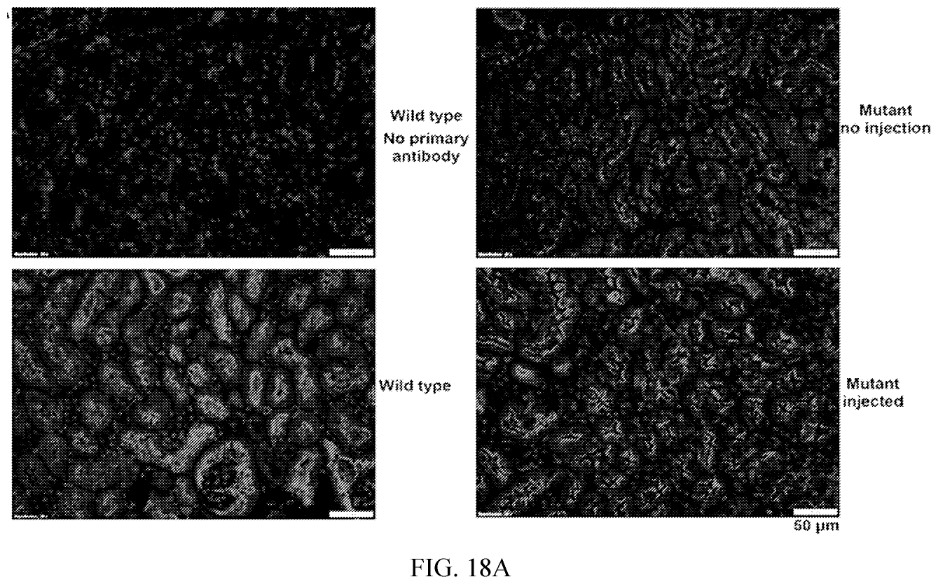

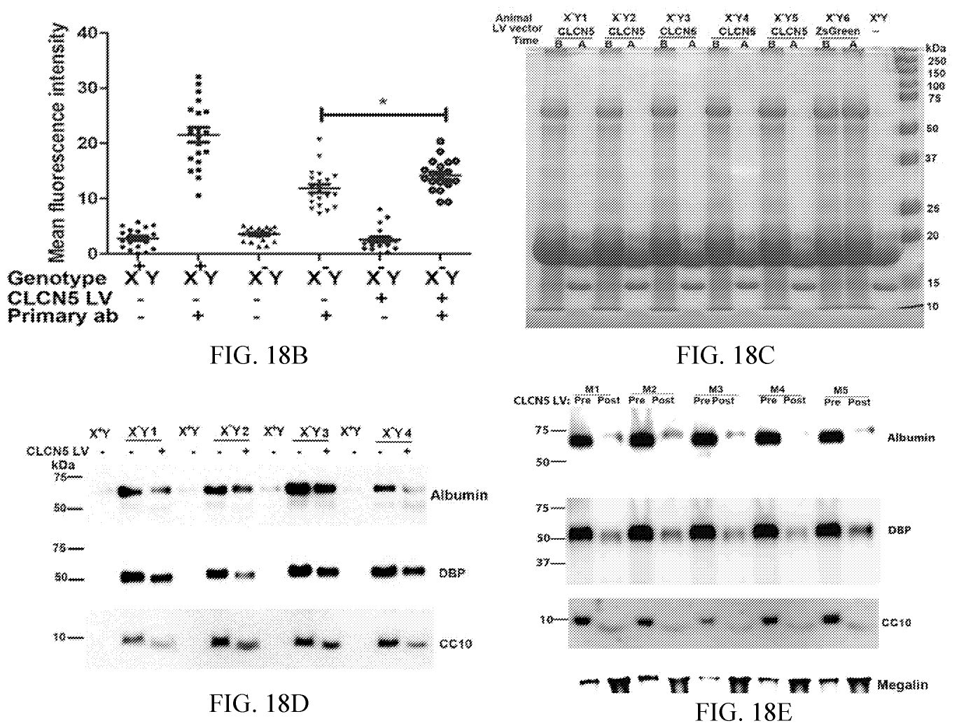

Studies were then conducted to assess whether intra-kidney delivery of the CLCN5-bearing lentivirus vector could correct CLCN5 expression in CLCN5 knockout mice (FIG. 10). It has been reported that CLCN5 deficiency causes the decrease of proteins involved in endocytosis, such as megalin and cubilin. We examined the expression of megalin and confirmed that megalin was decreased in the kidneys of mutant mice (FIG. 18A). We then delivered 280 ng p24 of CLCN5 LV into the kidneys of mutant mice and observed that in addition to restoring expression of CLCN5 (FIG. 18E), doing so also slightly increased the expression of megalin (FIG. 18A, 18B), although megalin expression was still lower than that in wild type mice.

Studies then determined whether delivering CLCN5 LV to the kidneys of mutant mice could improve phenotypes. Gene therapy experiments were then performed on the following three groups of male mutant mice. Group 1 received injection of 280 ng p24 ZsGreen LV into each kidney to serve as negative controls (5 mice); Group 2 received injection of 280 ng p24 CLCN5 LV into the left kidney (10 mice); Group 3 received injection of 280 ng p24 CLCN5 LV into each kidney (10 mice). One month after treatment, diuresis, urine protein and urine calcium levels of the three groups was examined. The DD1 phenotypes were not improved in ZsGreen LV injected mice, but were greatly improved in CLCN5 LV treated mice, regardless whether one kidney or both kidneys were treated (Table 4). After CLCN5 LV treatment, the diuresis and urine calcium values returned to normal levels (See Table 2 for normal values for male mice). Urine protein excretion after treatment reduced by 3-4 fold compared with before treatment although still higher than normal values.

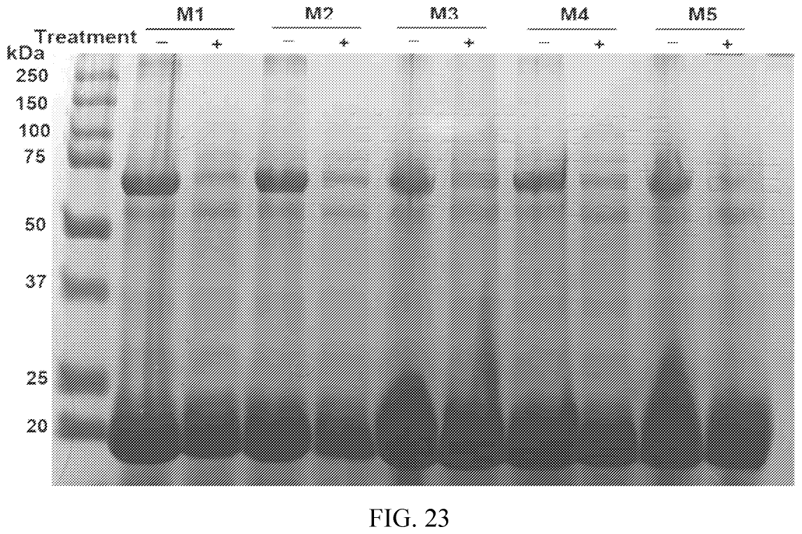

Consistent with reduction of total urine protein content after gene delivery in BCA assay, the intensities of the 61 kDa and <20 kDa bands in SDS-PAGE analysis were greatly reduced in urines of mice received CLCN5 LV but not ZsGreen LV injection (FIG. 18C, FIG. 23). Western blotting confirmed reduction in urine albumin, Vitamin D binding protein (DBP) and Club cell secretory protein (CC16, also called CC10) after delivering CLCN5 LV to the left kidney (FIG. 18B) or both kidneys (FIG. 18C). Equal volumes of urine samples were loaded in SDS-PAGE and Western blotting experiments. Considering the greatly reduced urine volume after CLCN5 gene therapy, the degree of urine protein reduction was even more dramatic. The ages of the CLCN5 LV treated mice varied from 25 days to 200 days, and all treated mice showed similar degree of improvement. Thus the timing of CLCN5 gene therapy seemed to have little influence on therapeutic effects.

### Example 4: Therapeutic Effects Lasted for Up to Four Months Following Gene Therapy

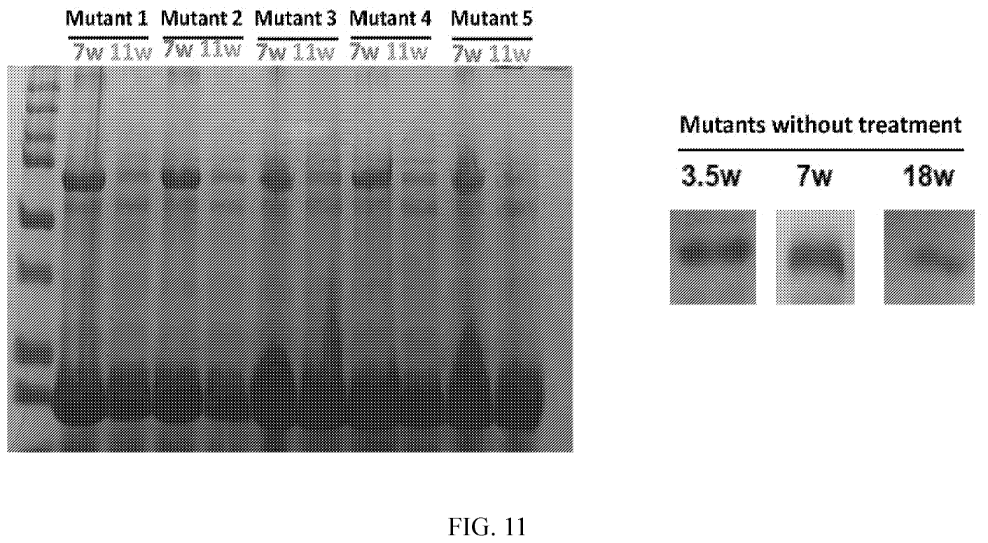

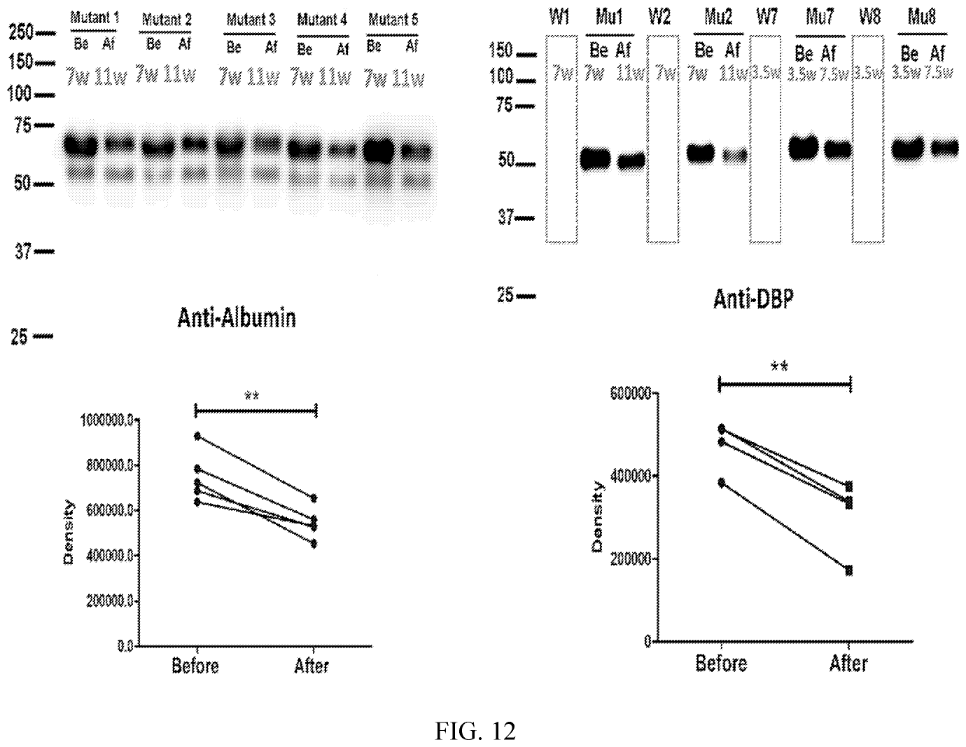

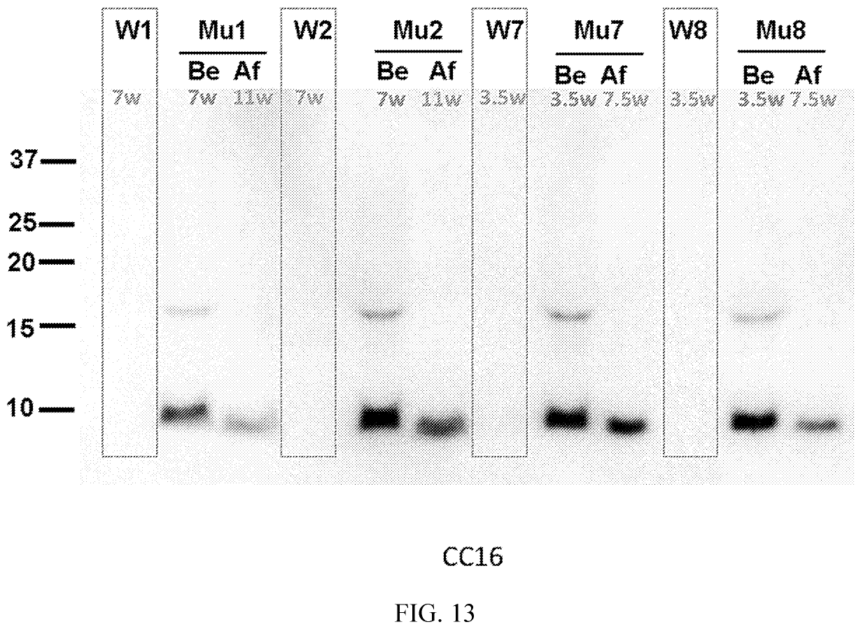

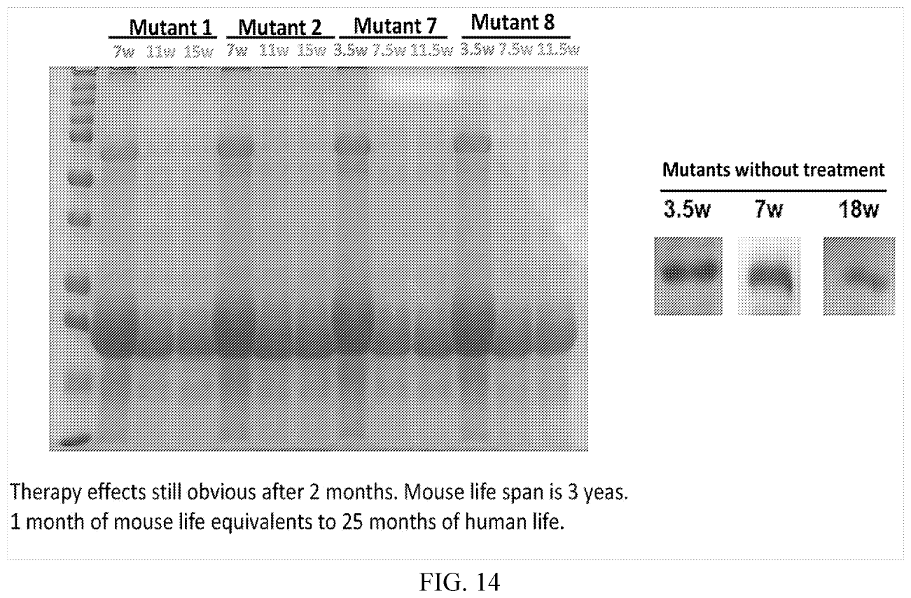

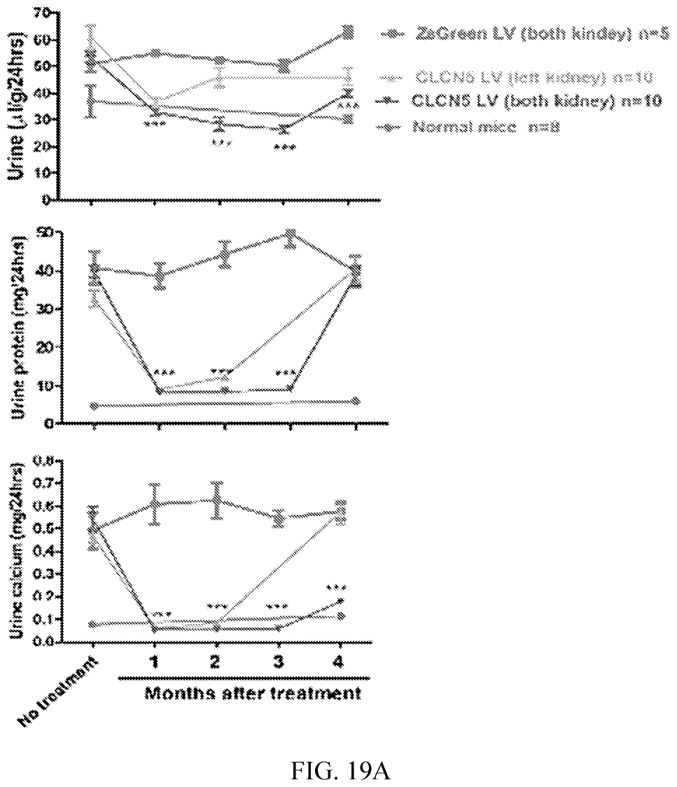

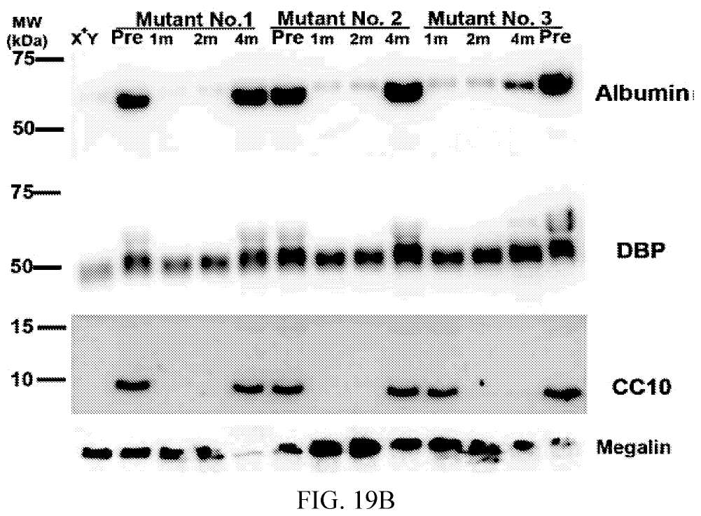

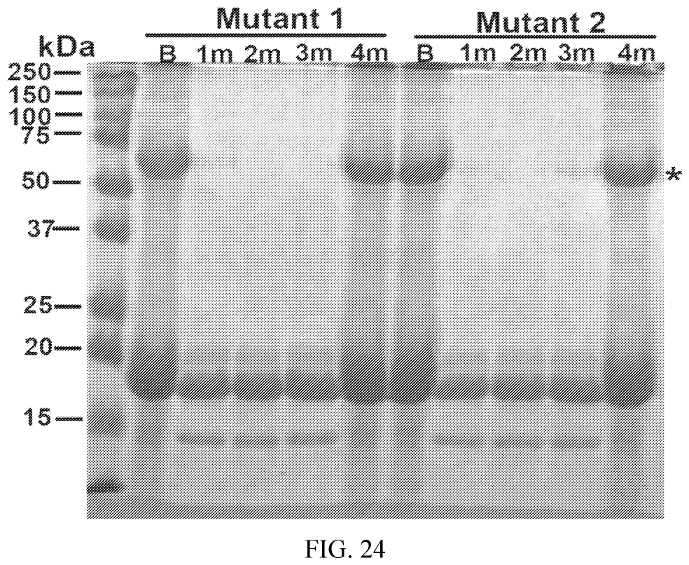

The urine produced by the animals was then monitored regularly for up to 4 months until the effects of the lentiviral vector dissipated. Kidney tissue was also harvested from selected animals at various timepoints for histological analysis of CLCN5 expression. Results demonstrated that even only one kidney was treated with lentivirus vectors (280 ng of p24), gene therapy greatly reduced urine protein secretion by mutant mice as assessed by SDS-PAGE (FIG. 11) as well as Western blot for albumin and vitamin D binding protein (FIG. 12). Likewise, Western blotting for CCL16 secretion also found a dramatic decrease in treated mutant animals (FIG. 13). The therapeutic effects were followed over time, and found to be detectable at one and two months after treatment, only disappearing at four months after therapy (FIG. 14 and Table 5). In mutant mice with one kidney treated, urine protein and urine calcium values returned to pre-treatment values 4 months after gene delivery, diuresis was also increased 1 month after treatment (FIG. 19A). In mutants with both kidneys treated, urine protein excretion returned to pre-treatment levels 4 months after treatment, whereas diuresis and calcium excretion were still lower than the pre-treatment levels 4 months after treatment (FIG. 19A), but returned to pre-treatment levels six months after therapy. Biochemical assay data was corroborated by SDS-PAGE and Western blotting analysis of urine proteins 4 months after gene therapy (FIG. 19B, FIG. 24). Given the three-year lifespan of the typical mouse, two months would represent approximately 50 months of a human lifespan.

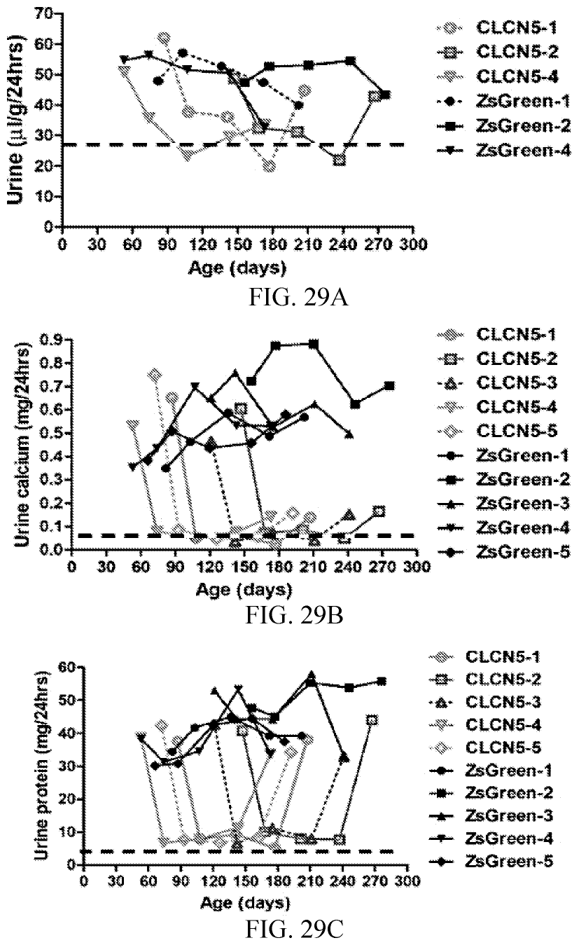

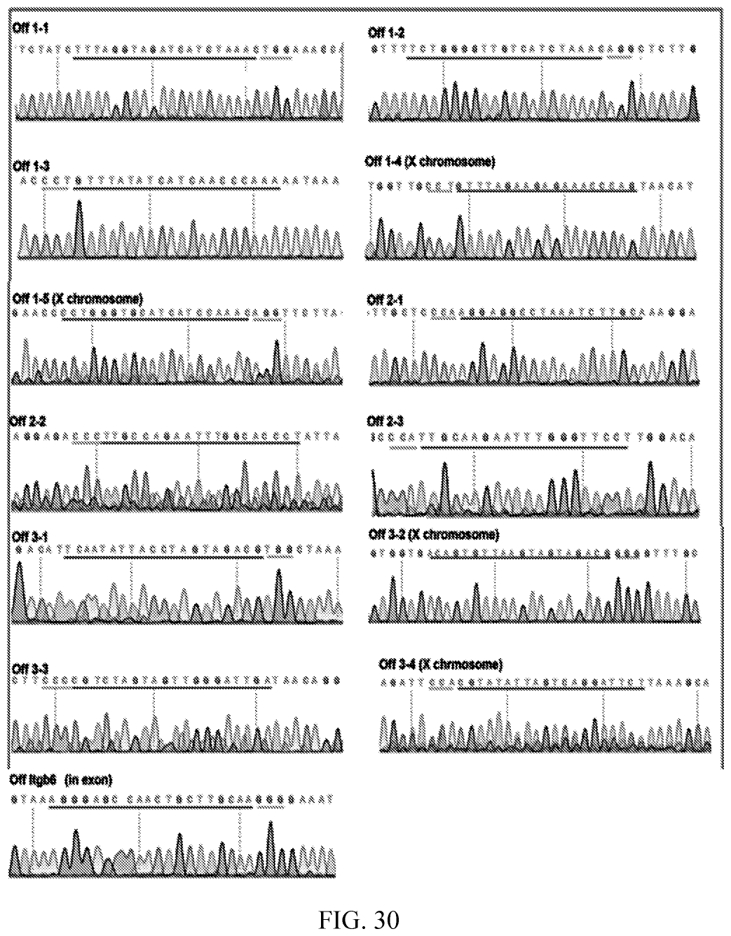

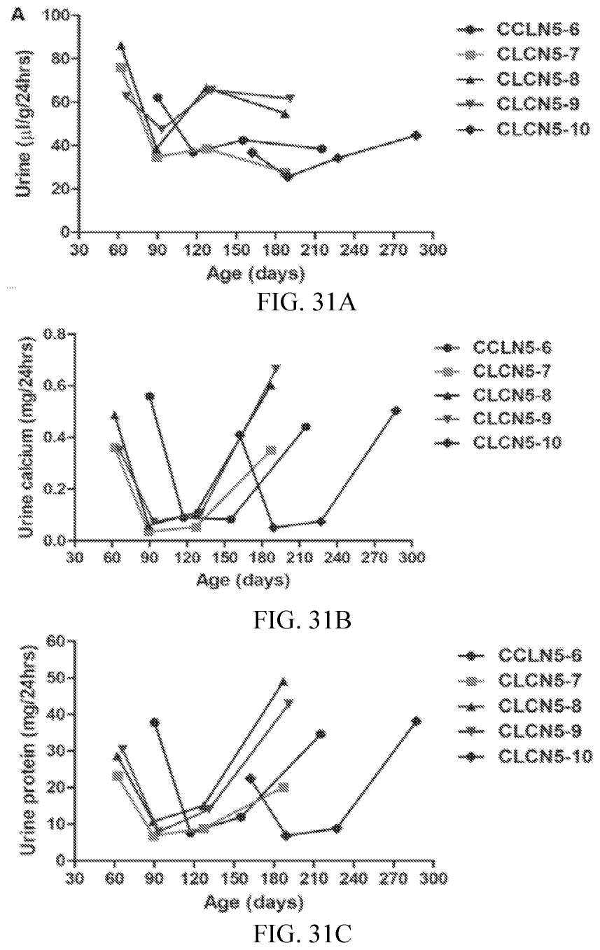

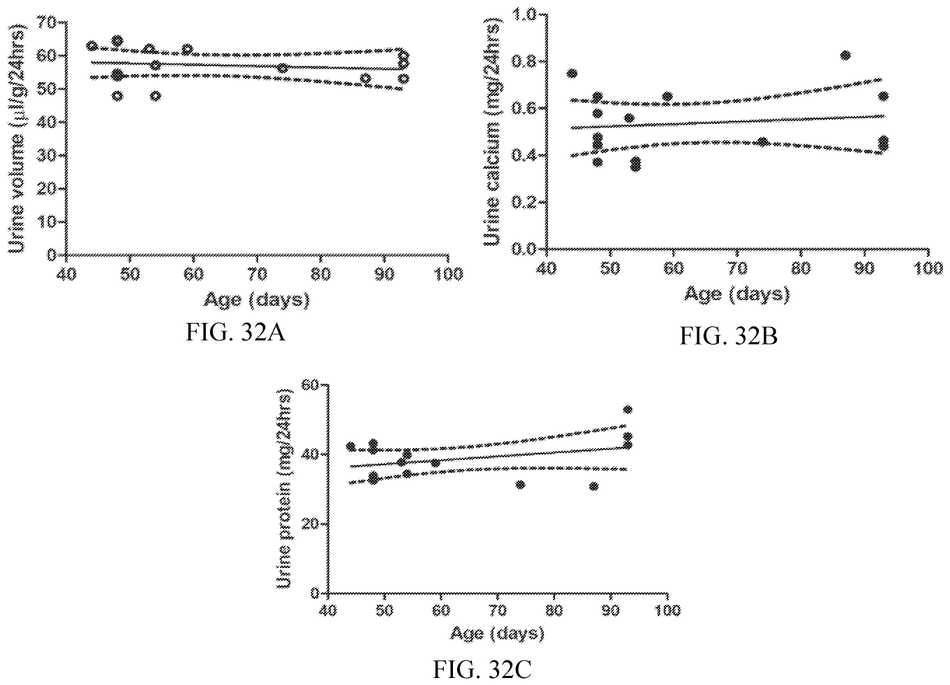

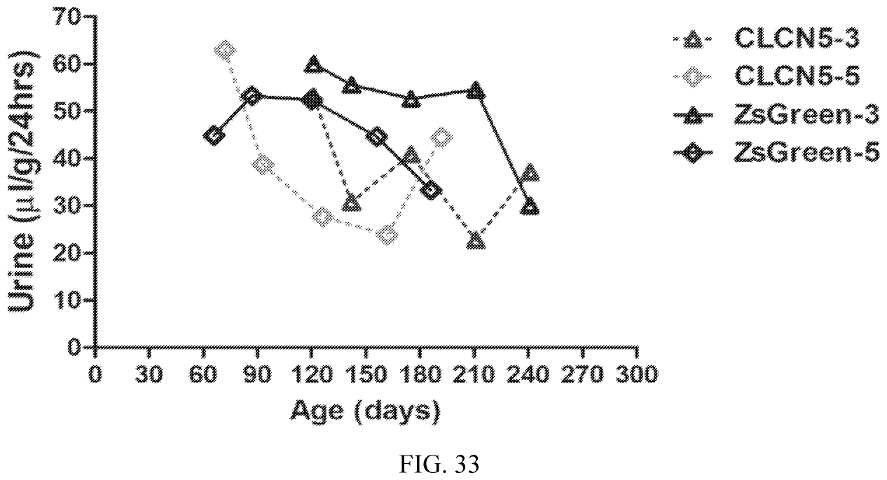

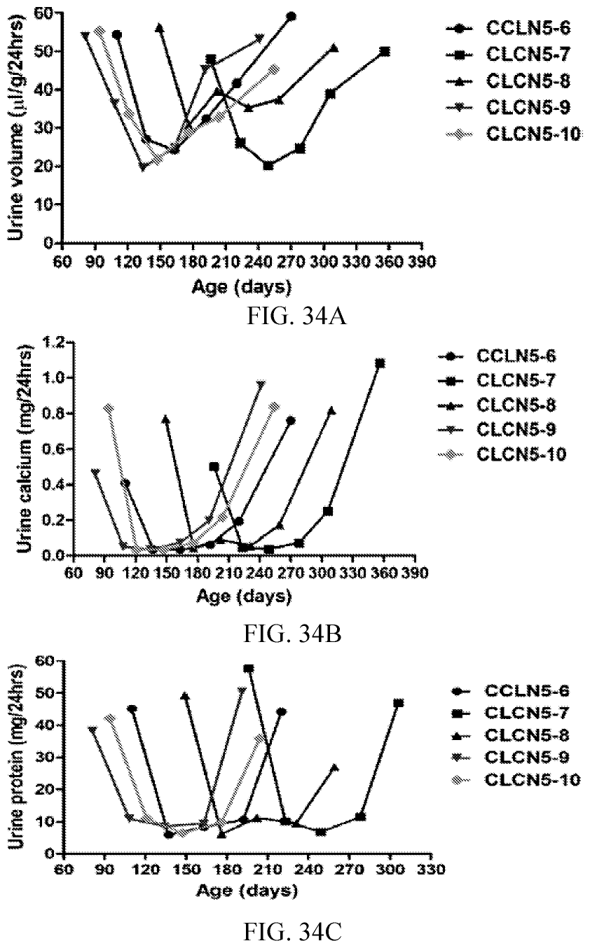

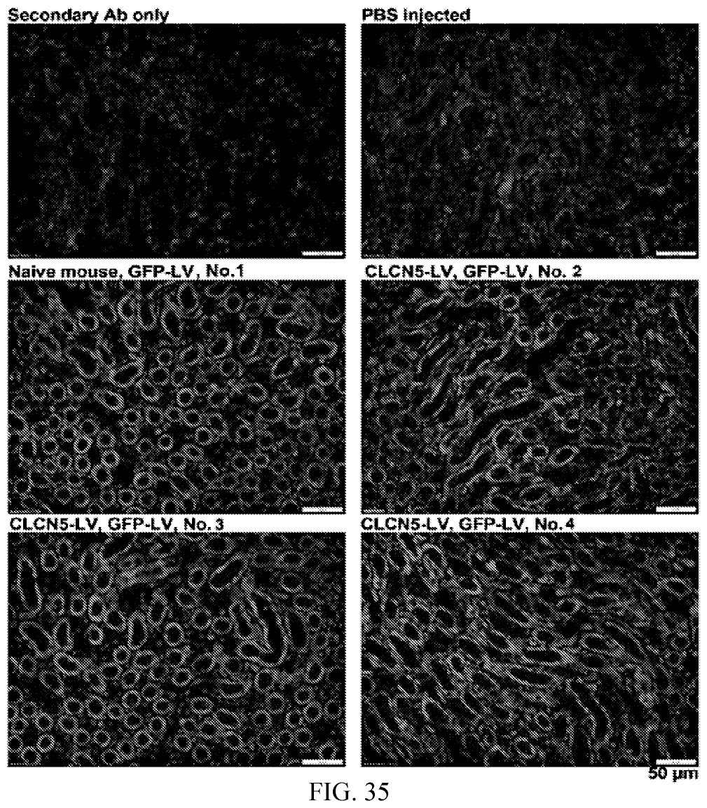

Related studies also delivered CLCN5 LV to the left kidneys of 5 mutant mice aged from 62-162 days. In every treated mouse a sharp decrease of urine protein and urine calcium excretion was observed one months after CLCN5 gene therapy (FIGS. 31A-31C). This decrease was not caused by aging of the mice, since aging did not cause significant decrease of these parameters (FIGS. 32A-32C). Another experiment was performed in which both kidneys of mutant mice aged from 53156 days were treated with CLCN5 LV, each CLCN5 LV-treated mouse had an age-matched mutant mouse treated with ZsGreen LV in both kidneys. One, two and three months after treatment, every single CLCN5 LV treated mouse showed greatly improved diuresis (FIG. 29A, FIG. 33), calciuria (FIG. 29B) and proteinuria (FIG. 29C), with levels close to those of wild type mice (dashed lines). On the contrary, no ZsGreen LV treated mice showed improvement in these parameters. Again, reduced urine protein one month after CLCN5 LV treatment was confirmed by SDS-PAGE (FIG. 18C) and Western blotting (FIG. 18E) analyses. Four months after both kidneys were treated with CLCN5 LV, diuresis and proteinuria returned to pre-treatment status (FIG. 29A, 29C), whereas calcinuria was still improved compared with pretreatment levels and ZsGreen LV treated mice (FIG. 29B). In addition, we treated both kidneys of 5 mutant mice aged from 81196 days with CLCN5 LV, again all of these mice responded to the therapy (FIGS. 34A-34C). In these mice urine calcinuria was still improved 4 months after treatment, but returned to pretreatment level 6 months after treatment (FIG. 34B). These data showed that the timing of CLCN5 gene therapy seemed to have little influence on therapeutic effects. Consistent with biochemical assays, SDS-PAGE (FIG. 24) and Western blotting (FIG. 19B) analyses of urine proteins also revealed that urine protein levels were reduced 1, 2 and 3 months after treatment, but returned to pre-treatment levels 4 months after treatment.

TABLE 5
Urine protein and calcium values of mutant mice before
and after one kidney being treated by gene therapy

| | Before | One month | Two months | Four months |
|---|---|---|---|---|
| | treatment | after treatment | after treatment | after treatment |
| Urine protein | 37.13 ± 2.08 | 8.71 ± 0.81*** | 10.60 ± 0.74*** | 37.82 ± 0.2.76 |
| (mg/24 hours) | | | | |
| Urine calcium | 0.87 ± 0.05 | 0.43 ± 0.03*** | 0.54 ± 0.03*** | 1.25 ± 0.08 |
| (mg/24 hours) | | | | |

***indicates p < 0.0001 compared with values before treatment. Ten mice were included for each group.

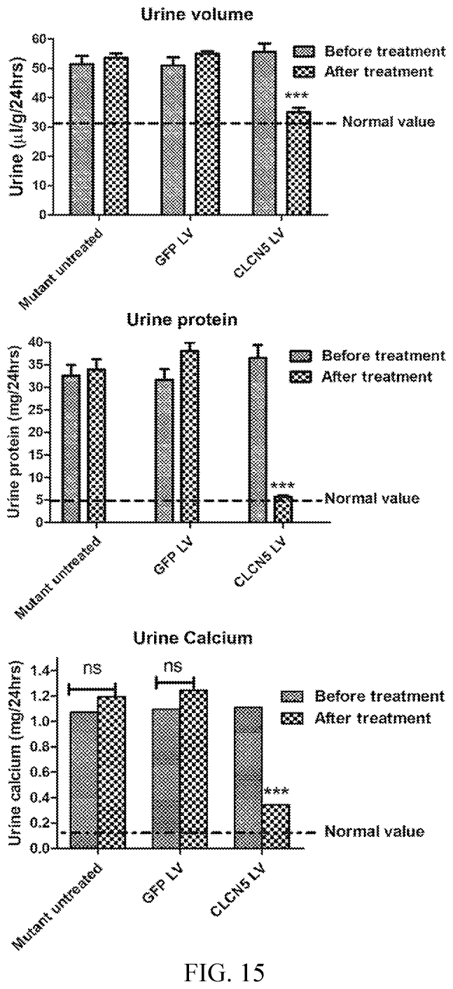

Studies were then conducted in which both kidneys of mutant mice were treated with a single injection of CLCN5 lentiviral vector (280 ng of p24). Untreated mutant mice and mutant mice treated with GFP-expressing lentiviral vectors were used as controls. One month after treatment, the urine volume and urine protein levels were found to be almost restored to normal levels, and urine calcium levels were four times lower than those of untreated mutant mice, although still higher than that of normal mice (FIG. 15).

### Example 5: Immune Rejection Underlay the Loss of Therapeutic Effects

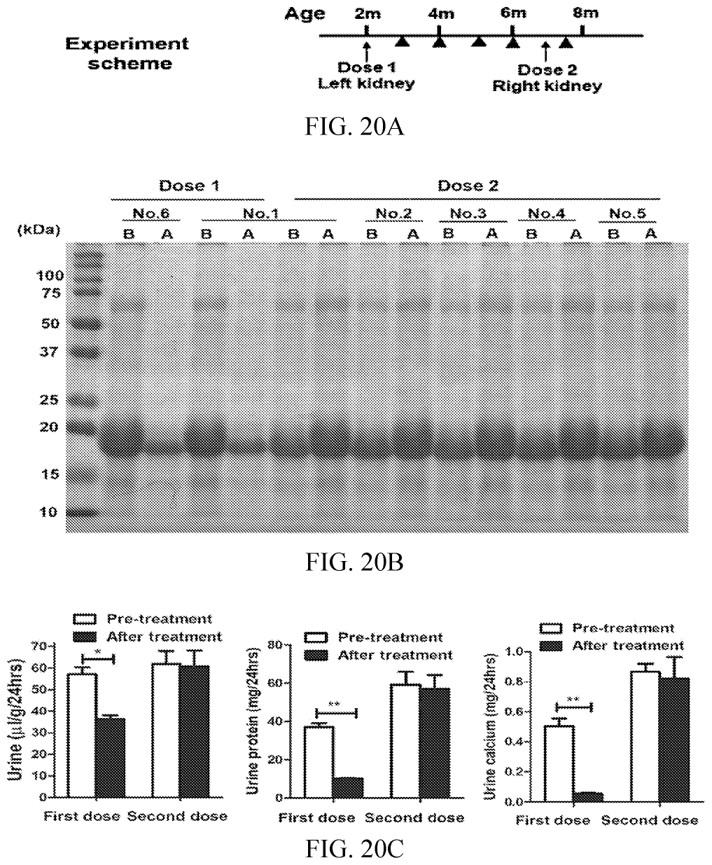

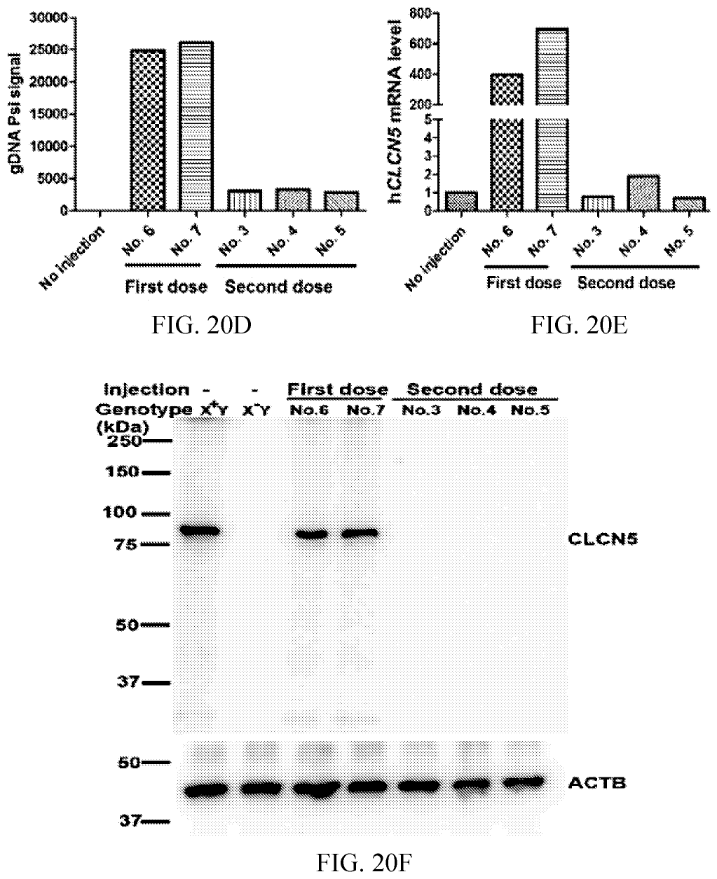

Without wishing to be bound by theory, there could be several possible explanations for the loss of therapeutic effects four months after treatment: 1) promoter silencing; 2) natural epithelial cell aging and replacement; and 3) immune rejection of the CLCN5-expressing cells. A series of experiments was then performed in order to find the most likely mechanisms. A second dose of LV was then delivered to the untreated right kidney of mutant mice 5 months after receiving a first dose of CLCN5 LV in the left kidney (FIG. 20A), when the therapeutic effects of the first dose were lost. If the loss of therapeutic effects was caused by promoter silencing or natural aging of treated cells, we should observe therapeutic effects after receiving the second dose. If it was caused by immune rejection, therapeutic effects should not be observed after the second dose.

As positive controls, naïve mutants were similarly treated in order to validate the LV and the delivery procedure (FIG. 20, animal No. 6 and 7). Urine samples were collected 15 days after vector delivery and observed clearly reduced urine protein after therapy in naïve mice (FIG. 20B, mouse No. 6), demonstrating the success of the procedure and the functionality of the vectors. However, urine protein reduction was not observed in any of the five pre-treated mice (animal No. 1-5), although in these mice urine protein was obviously reduced after the first dose of CLCN5 LV (FIG. 20B). Diuresis, urine protein, and calcium excretion were only improved after the first dose but not the second one (FIG. 20C). These data suggested that immune rejection was most likely the major underlying mechanism.

LV DNA integration, human CLCN5 mRNA expression and CLCN5 protein expression was also examined in the injected kidneys. LV DNA (Psi signal) (FIG. 20D), human CLCN5 mRNA (FIG. 20E) and CLCN5 protein (FIG. 20F) could be detected in kidneys of naïve mice (animal No. 6 and 7), but were greatly reduced or undetectable in kidneys of pre-treated mice (animals No. 3, 4 and 5). In contrast to the lack of CLCN5 expression following delivering a second CLCN5 LV injection to mice pretreated with CLCN5 LV, delivering GFP LV to mice pretreated with CLCN5 LV resulted in robust GFP expression (FIG. 35, CLCN5-LV, GFP-LV, No. 2-4). These data were consistent with the observation of gene therapy effects one months after the first dose but not after the second dose, further confirming immune rejection.

### Example 6: Selected Discussion

In summation, and without wishing to be bound by theory, these results demonstrate that mouse models of Dent disease of the current invention can be created in which CLCN5 expression is ablated, resulting in phenotypic and functional consequences that mirror the clinical manifestations of Dent disease in human patients. Notably, the Dent disease mouse model of the current invention demonstrates much more obvious phenotypes than current Dent disease mouse models created through homologous recombination. One unexpected phenotype was the partial embryonic or perinatal lethality of the mutant mice in C57/BL6 background. This observation suggests that CLCN5 gene may function during early development, consistent with observation of CLCN5 expression during embryonic development and in organs other than the kidney. In addition, mutant mice showed more severe proteinuria and hypercalciuria compared with published models. There are no other predicted genes (including non-coding genes) within 40 kilo bps surrounding the deleted region. Thus the observed phenotypes were the results of deleting 95% of the CLCN5 coding region, which eliminated the possibility of expressing a partially functional CLCN5 protein. Thus these null mutants may be useful to study the physiological consequences of complete lack of CLCN5 protein.

The data disclosed herein shows that gene supplementary therapy can be an effective treatment option for DD1. CLCN5 LV vectors to were delivered to 22 mutant mice (10 mice were treated in one kidney and 12 mice were treated in both kidneys) and 100% of the treated mice showed significant improvement in all parameters examined: diuresis, proteinuria and hypercalciuria. After treatment, the diuresis and urine calcium levels were restored to normal values, whereas the urine protein values reduced to 20% of pre-treatment values, although still 80% higher than normal values. Thus gene therapy is very effective in ameliorating the symptoms of DD1. Another interesting observation is that mice as young as 25 days and as old as 200 days (6.5 months) responded similarly to the therapy, indicating that the timing of gene therapy is not critical in this disease. This observation is relevant in clinical applications since not only young patients can benefit from the gene therapy.

CLCN5 is also expressed in the intestinal epithelium, and one study raised the possible role of intestinal calcium absorption in hypercalciuria of CLCN5 deficient mice. We delivered the CLCN5 LV into the kidney by retrograde ureter injection and completely restored urine calcium level in mutant mice. These data suggest that CLCN5 expressed in the kidney plays a major role in calcium maintenance.

Frameshift and nonsense mutations account for 29% and 17.5% of all DD1-causing mutations, and these mutations are likely to result in the expression of a truncated CLCN5 protein or the absence of the protein entirely, as seen in the model mice of the present disclosure. These data suggest that gene supplementary therapy most likely will benefit these patients. About 33% DD1-causing mutations are missense ones, which express unstable proteins, dislocated proteins or dysfunctional proteins. Gene therapy may benefit some of those subjects expressing unstable or dislocated CLCN5 proteins. It remains to be determined to what extent gene therapy will benefit those subjects expressing a malfunctioned CLCN5, since CLCN5 most likely forms a homodimer, and the endogenous malfunctioned CLCN5 protein might interfere with the function of the exogenous CLCN5 protein.

In the studies of the present disclosure, the gene therapy effects lasted for up to 4 months. Consistent with the observation that gene therapy completely normalized the urine calcium level but not the urine protein level, the beneficiary effect on hypercalciuria lasted longer than on proteinuria. Immune responses seemed to be the major mechanism underlying the loss of gene therapy effects, which was supported by the lack of therapy effects after delivering a second dose of LV to the pre-treated mice. Attenuated gene therapy effects was first observed two months after gene delivery.

Immune responses to transgene products have been observed before. Since the mutant mice do not express CLCN5 at all, the constitutively expressed human CLCN5 protein expressed from the LV vector is expected to induce an adaptive immune response. It remains to be determined whether less severe immune responses will be observed in subjects expressing an unstable or malfunctioned CLCN5 proteins.

The gradual loss of therapeutic effects due to host immune responses suggests the importance of suppressing host immune responses to achieve long-term gene therapy effects. There are several strategies to help minimizing immune responses. One is to use tissue-specific promoters to avoid expression of the transgene in dendritic cells (DCs), which are the mediator of adaptive immune responses. In this study we used the EF1 alpha promoter active in essentially all cells for proof of concept. Since proximal tubules are the main location of reabsorbing, using tubule proximal cell specific promoters such as those for Npt2a or Sgt12 may help to reduce immune responses.

### Enumerated Embodiments

The following enumerated embodiments are provided, the numbering of which is not to be construed as designating levels of importance.

Embodiment 1 provides a method for treating Dent disease in a subject in need thereof, the method comprising administering to the subject an effective amount of a nucleic acid vector encoding a CLCN5 protein, thereby treating the disease.

Embodiment 2 provides the method of claim 1, wherein the nucleic acid vector is a lentiviral vector.

Embodiment 3 provides the method of claim 1, wherein the nucleic acid vector is operably linked to a promoter that drives the expression of the CLCN5 protein.

Embodiment 4 provides the method of claim 3, wherein the promoter is a constitutive promoter.

Embodiment 5 provides the method of claim 4, wherein the promoter is an EF-1α promoter.

Embodiment 6 provides the method of claim 3, wherein the promoter is a tissue-specific promoter.

Embodiment 7 provides the method of claim 6, wherein the tissue-specific promoter is specific for renal tubule proximal cells.

Embodiment 8 provides the method of claim 7, wherein the tissue specific promoter is selected from the group consisting of Npt2a and Sgt12.

Embodiment 9 provides the method of claim 2, wherein the lentiviral vector is encoded by the nucleic acid sequence set forth in SEQ ID NO. 1.

Embodiment 10 provides the method of claim 1, wherein the administration is delivered locally to the kidney.

Embodiment 11 provides the method of claim 10, wherein the local kidney administration is delivered by retrograde ureteral injection.

Embodiment 12 provides a method for correcting a mutation in the CLCN5 gene in a cell, said method comprising contacting the cell with a nucleic acid vector encoding a functional CLCN5 protein.

Embodiment 13 provides the method of claim 12, wherein the nucleic acid vector is a lentiviral vector.

Embodiment 14 provides the method of claim 12, wherein the nucleic acid vector is operably linked to a promoter that drives expression of the CLCN5 protein.

Embodiment 15 provides the method of claim 14, wherein the promoter is a constitutive promoter.

Embodiment 16 provides the method of claim 15, wherein the promoter is an EF-1α promoter.

Embodiment 17 provides the method of claim 14, wherein the promoter is a tissue-specific promoter.

Embodiment 18 provides the method of claim 17, wherein the tissue-specific promoter is specific for renal tubule proximal cells.

Embodiment 19 provides the method of claim 18, wherein the tissue specific promoter is selected from the group consisting of Npt2a and Sgt12.

Embodiment 20 provides the method of claim 13, wherein the lentiviral vector is encoded by the nucleic acid sequence set forth in SEQ ID NO: 1.

Embodiment 21 provides a pharmaceutical composition comprising a nucleic acid vector encoding a CLCN5 protein and a pharmaceutically acceptable carrier.

Embodiment 22 provides the pharmaceutical composition of claim 21, wherein the nucleic acid vector is a lentiviral vector.

Embodiment 23 provides the pharmaceutical composition of claim 22, wherein the lentiviral vector is encoded by a nucleic acid sequence set forth in SEQ ID NO: 1.

Embodiment 24 provides a mouse model of type 1 Dent disease, wherein the mouse comprises one or more mutation in the CLCN5 gene in the mouse.

Embodiment 25 provides the mouse model of claim 24, wherein the one or more mutations is a deletion.

Embodiment 26 provides the mouse model of claim 25, wherein the deletion affects exon 3, exon 4, exon 5, exon 6, exon 7, exon 8, exon 9, exon 10, and exon 11 of the CLCN5 gene.

Embodiment 27 provides the mouse model of claim 24, wherein the one or more CLCN5 mutations result in a non-functional CLCN5 protein.

Embodiment 28 provides the mouse model of claim 24, wherein the breeding of experimental animals involves a sire and dam being of different strains.

Embodiment 29 provides the mouse model of claim 28, wherein the dam is a heterozygous for the CLCN5 mutation and the sire is wildtype.

Embodiment 30 provides the mouse model of claim 28, wherein the sire is of the FVB background.

Embodiment 31 provides the mouse model of claim 28, wherein the dam is of the C57BL/6 background.

### OTHER EMBODIMENTS

The recitation of a listing of elements in any definition of a variable herein includes definitions of that variable as any single element or combination (or sub combination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method for treating Dent disease in a subject in need thereof, the method comprising administering to the subject an effective amount of a nucleic acid vector encoding a CLCN5 protein, thereby treating the disease.

2. The method of claim 1, wherein the nucleic acid vector is a lentiviral vector.

3. The method of claim 1, wherein the nucleic acid vector is operably linked to a promoter that drives the expression of the CLCN5 protein.

4. The method of claim 3, wherein the promoter is a constitutive promoter.

5. The method of claim 4, wherein the promoter is an EF-1α promoter.

6. The method of claim 3, wherein the promoter is a tissue-specific promoter.

7. The method of claim 6, wherein the tissue-specific promoter is specific for renal tubule proximal cells.

8. The method of claim 7, wherein the tissue specific promoter is selected from the group consisting of Npt2a and Sgt12.

9. The method of claim 2, wherein the lentiviral vector is encoded by the nucleic acid sequence set forth in SEQ ID NO. 1.

10. The method of claim 1, wherein the administration is delivered locally to the kidney.

11. The method of claim 10, wherein the local kidney administration is delivered by retrograde ureteral injection.

12. A method for correcting a mutation in the CLCN5 gene in a cell, said method comprising contacting the cell with a nucleic acid vector encoding a functional CLCN5 protein.

13. The method of claim 12, wherein the nucleic acid vector is a lentiviral vector.

14. The method of claim 12, wherein the nucleic acid vector is operably linked to a promoter that drives expression of the CLCN5 protein.

15. The method of claim 14, wherein the promoter is a constitutive promoter.

16. The method of claim 15, wherein the promoter is an EF-1α promoter.

17. The method of claim 14, wherein the promoter is a tissue-specific promoter.

18. The method of claim 17, wherein the tissue-specific promoter is specific for renal tubule proximal cells.

19. The method of claim 18, wherein the tissue specific promoter is selected from the group consisting of Npt2a and Sgt12.

20. The method of claim 13, wherein the lentiviral vector is encoded by the nucleic acid sequence set forth in SEQ ID NO: 1.

21. A pharmaceutical composition comprising a nucleic acid vector encoding a CLCN5 protein and a pharmaceutically acceptable carrier.

22. The pharmaceutical composition of claim 21, wherein the nucleic acid vector is a lentiviral vector.

23. The pharmaceutical composition of claim 22, wherein the lentiviral vector is encoded by a nucleic acid sequence set forth in SEQ ID NO: 1.

24. A mouse model of type 1 Dent disease, wherein the mouse comprises one or more mutation in the CLCN5 gene in the mouse.

25. The mouse model of claim 24, wherein the one or more mutations is a deletion.

26. The mouse model of claim 25, wherein the deletion affects exon 3, exon 4, exon 5, exon 6, exon 7, exon 8, exon 9, exon 10, and exon 11 of the CLCN5 gene.

27. The mouse model of claim 24, wherein the one or more CLCN5 mutations result in a non-functional CLCN5 protein.

28. The mouse model of claim 24, wherein the breeding of experimental animals involves a sire and dam being of different strains.

29. The mouse model of claim 28, wherein the dam is a heterozygous for the CLCN5 mutation and the sire is wildtype.

30. The mouse model of claim 28, wherein the sire is of the FVB background.

31. The mouse model of claim 28, wherein the dam is of the C57BL/6 background.


SEQUENCE LISTING (20 sequences)

SEQ ID NO: 1 - DNA, 11359 bp, Artificial Sequence
  hCLCN5 vector
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaattcactttgaattaattcaagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaggggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaacacgctaccggtctcgagccaccatggatttcctggaggaaccaataccaggtgtaggaacatatgacgatttcaatactatagactgggtgcgagagaaatcacgcgatcgagacagacaccgggagatcacgaataagtctaaggaatctacctgggccctcattcacagtgtgtcagacgcttttagcggatggctgcttatgcttctgattggacttcttagtggtagtttggcgggcctgatagacattagcgcgcactggatgactgatcttaaagaaggcatatgcacggggggattttggttcaaccacgaacattgctgctggaactccgagcatgtgacattcgaggagagggacaagtgccccgagtggaatagttggagccaactgataatttctacagatgagggggcttttgcctatatagttaattatttcatgtatgttttgtgggccctcctcttcgccttcctcgcggtatccctcgttaaggtctttgccccatatgcctgtggctctggtattccagaaataaaaactatcctttctggatttataatcaggggatatctgggcaagtggacgttggtcattaagacaatcacccttgtccttgctgtatcttcagggttgtccttgggcaaagagggtcctctcgttcacgtagcttgctgctgtgggaacatcctttgccattgtttcaataaatataggaagaacgaagcaaagcgccgagaagttctgagcgcagcagcggccgcaggtgtcagtgttgccttcggggctcctataggaggggtactgtttagtctcgaagaagtgtcatattactttcctctcaagacactgtggaggtccttttttgcagccctggtcgcggcttttactctgcgctctattaatccttttggaaacagcagacttgtgctgttctacgtcgaattccacaccccgtggcatttgtttgaactcgtaccctttattttgctggggattttcggtggattgtggggtgctctgttcatacgcactaacattgcgtggtgccggaagaggaagactactcagttgggcaaatacccagttattgaggtcctcgtcgttacagctatcacagcaattcttgcgttccccaacgagtacacacggatgtctacatccgaactgattagcgaactgttcaatgattgtgggctcttggactcctcaaaactgtgcgattatgaaaatcgatttaatacatcaaagggcggagaacttcccgatcggccggctggagtgggagtatactccgctatgtggcagctggcgttgacgctcatactcaaaatcgtcattaccatattcacttttggaatgaagattccctcaggtctctttatccctagtatggcagttggtgcgattgcgggacggctcctgggcgttggcatggagcagctggcttattaccatcaggagtggaccgtattcaatagctggtgctctcagggcgctgattgcatcacaccaggcctgtatgccatggtaggcgctgctgcttgtcttggaggggtgactaggatgacggtttctctcgtcgtgataatgttcgagcttactgggggtcttgagtacattgtgcccctgatggcggcggcaatgacatccaaatgggtggcggatgcgttgggtagggaagggatatacgatgcacatattcgccttaatggctacccatttttggaggctaaggaagaatttgcacataaaactctcgccatggatgttatgaaaccgagacgaaacgacccattgcttacagtacttacacaggattccatgaccgttgaggacgtggaaacaataatatctgaaacaacttatagtggctttcccgtcgtcgtatcccgagaatcacaaaggttggtaggattcgtgctgcgacgcgacctgatcatatccatagaaaacgcacgcaagaagcaagacggggtagtgtccacgtctataatttatttcaccgagcatagccctcccttgcctccatatactccgcctacactgaaacttcgaaacatcctcgatttgtctccttttacagtaaccgaccttactccaatggaaatcgtagtagacatatttagaaagcttggattgaggcaatgcctggttacccacaacggtcggttgctcgggataataacgaagaaggacgtactcaaacatatagcacaaatggcaaaccaggacccggattcaatcttgttcaactagtctagagttaacatcgagggatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgagacctggaaaaacatggagcaatcacaagtagcaacacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - DNA, 2241 bp, Artificial Sequence
  hCLCN5 cDNA
atggatttcctggaggaaccaataccaggtgtaggaacatatgacgatttcaatactatagactgggtgcgagagaaatcacgcgatcgagacagacaccgggagatcacgaataagtctaaggaatctacctgggccctcattcacagtgtgtcagacgcttttagcggatggctgcttatgcttctgattggacttcttagtggtagtttggcgggcctgatagacattagcgcgcactggatgactgatcttaaagaaggcatatgcacggggggattttggttcaaccacgaacattgctgctggaactccgagcatgtgacattcgaggagagggacaagtgccccgagtggaatagttggagccaactgataatttctacagatgagggggcttttgcctatatagttaattatttcatgtatgttttgtgggccctcctcttcgccttcctcgcggtatccctcgttaaggtctttgccccatatgcctgtggctctggtattccagaaataaaaactatcctttctggatttataatcaggggatatctgggcaagtggacgttggtcattaagacaatcacccttgtccttgctgtatcttcagggttgtccttgggcaaagagggtcctctcgttcacgtagcttgctgctgtgggaacatcctttgccattgtttcaataaatataggaagaacgaagcaaagcgccgagaagttctgagcgcagcagcggccgcaggtgtcagtgttgccttcggggctcctataggaggggtactgtttagtctcgaagaagtgtcatattactttcctctcaagacactgtggaggtccttttttgcagccctggtcgcggcttttactctgcgctctattaatccttttggaaacagcagacttgtgctgttctacgtcgaattccacaccccgtggcatttgtttgaactcgtaccctttattttgctggggattttcggtggattgtggggtgctctgttcatacgcactaacattgcgtggtgccggaagaggaagactactcagttgggcaaatacccagttattgaggtcctcgtcgttacagctatcacagcaattcttgcgttccccaacgagtacacacggatgtctacatccgaactgattagcgaactgttcaatgattgtgggctcttggactcctcaaaactgtgcgattatgaaaatcgatttaatacatcaaagggcggagaacttcccgatcggccggctggagtgggagtatactccgctatgtggcagctggcgttgacgctcatactcaaaatcgtcattaccatattcacttttggaatgaagattccctcaggtctctttatccctagtatggcagttggtgcgattgcgggacggctcctgggcgttggcatggagcagctggcttattaccatcaggagtggaccgtattcaatagctggtgctctcagggcgctgattgcatcacaccaggcctgtatgccatggtaggcgctgctgcttgtcttggaggggtgactaggatgacggtttctctcgtcgtgataatgttcgagcttactgggggtcttgagtacattgtgcccctgatggcggcggcaatgacatccaaatgggtggcggatgcgttgggtagggaagggatatacgatgcacatattcgccttaatggctacccatttttggaggctaaggaagaatttgcacataaaactctcgccatggatgttatgaaaccgagacgaaacgacccattgcttacagtacttacacaggattccatgaccgttgaggacgtggaaacaataatatctgaaacaacttatagtggctttcccgtcgtcgtatcccgagaatcacaaaggttggtaggattcgtgctgcgacgcgacctgatcatatccatagaaaacgcacgcaagaagcaagacggggtagtgtccacgtctataatttatttcaccgagcatagccctcccttgcctccatatactccgcctacactgaaacttcgaaacatcctcgatttgtctccttttacagtaaccgaccttactccaatggaaatcgtagtagacatatttagaaagcttggattgaggcaatgcctggttacccacaacggtcggttgctcgggataataacgaagaaggacgtactcaaacatatagcacaaatggcaaaccaggacccggattcaatcttgttcaactag

SEQ ID NO: 3 - protein, 746 aa, Artificial Sequence
  hCLCN5 protein
MDFLEEPIPGVGTYDDFNTIDWVREKSRDRDRHREITNKSKESTWALIHSVSDAFSGWLLMLLIGLLSGSLAGLIDISAHWMTDLKEGICTGGFWFNHEHCCWNSEHVTFEERDKCPEWNSWSQLIISTDEGAFAYIVNYFMYVLWALLFAFLAVSLVKVFAPYACGSGIPEIKTILSGFIIRGYLGKWTLVIKTITLVLAVSSGLSLGKEGPLVHVACCCGNILCHCFNKYRKNEAKRREVLSAAAAAGVSVAFGAPIGGVLFSLEEVSYYFPLKTLWRSFFAALVAAFTLRSINPFGNSRLVLFYVEFHTPWHLFELVPFILLGIFGGLWGALFIRTNIAWCRKRKTTQLGKYPVIEVLVVTAITAILAFPNEYTRMSTSELISELFNDCGLLDSSKLCDYENRFNTSKGGELPDRPAGVGVYSAMWQLALTLILKIVITIFTFGMKIPSGLFIPSMAVGAIAGRLLGVGMEQLAYYHQEWTVFNSWCSQGADCITPGLYAMVGAAACLGGVTRMTVSLVVIMFELTGGLEYIVPLMAAAMTSKWVADALGREGIYDAHIRLNGYPFLEAKEEFAHKTLAMDVMKPRRNDPLLTVLTQDSMTVEDVETIISETTYSGFPVVVSRESQRLVGFVLRRDLIISIENARKKQDGVVSTSIIYFTEHSPPLPPYTPPTLKLRNILDLSPFTVTDLTPMEIVVDIFRKLGLRQCLVTHNGRLLGIITKKDVLKHIAQMANQDPDSILFN

SEQ ID NO: 4 - DNA, 1163 bp, Artificial Sequence
  EF1alpha promoter
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaggggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 5 - DNA, 20 bp, Artificial Sequence
  hCLCN5-F(cDNA) primer
tctcgccatggatgttatga

SEQ ID NO: 6 - DNA, 20 bp, Artificial Sequence
  hCLCN5-R(cDNA) primer
tcttgcgtgcgttttctatg

SEQ ID NO: 7 - DNA, 20 bp, Artificial Sequence
  mCLCN5-EF primer
ccctggtgtagggacctatg

SEQ ID NO: 8 - DNA, 20 bp, Artificial Sequence
  mCLCN5-ER primer
cagaattccagcaacagtgc

SEQ ID NO: 9 - DNA, 20 bp, Artificial Sequence
  CLCN5-KF2 primer
aagggacagtcatggtctgg

SEQ ID NO: 10 - DNA, 20 bp, Artificial Sequence
  CLCN5-KR2 primer
caatggcctgttgtgcatac

SEQ ID NO: 11 - DNA, 20 bp, Artificial Sequence
  CLCN5-W2 primer
ctgggtttcatgcatttgtg

SEQ ID NO: 12 - DNA, 17 bp, Artificial Sequence
  Psi-F primer
tctcgacgcaggactcg

SEQ ID NO: 13 - DNA, 18 bp, Artificial Sequence
  Psi-R primer
tactgacgctctcgcacc

SEQ ID NO: 14 - RNA, 20 nt, Artificial Sequence
  mCLCN5 intron 2 gRNA
ucuggguugaucaucuaaac

SEQ ID NO: 15 - RNA, 20 nt, Artificial Sequence
  mCLCN5 intron 5 gRNA
agggggccgaauucuugcaa

SEQ ID NO: 16 - RNA, 20 nt, Artificial Sequence
  mCLCN5 exon 12 gRNA
gcaaugcuaacuaguagacg

SEQ ID NO: 17 - DNA, 48 bp, Artificial Sequence
  Fig 2b
ggctagcctggaaggaactctctgggttgttagaaatctgttagtagc

SEQ ID NO: 18 - DNA, 32 bp, Artificial Sequence
  Fig 7
atggacttcttggaggagccaatccctggtgt

SEQ ID NO: 19 - protein, 11 aa, Artificial Sequence
  Fig 7
MDFLEEPIPGV

SEQ ID NO: 20 - DNA, 32 bp, Artificial Sequence
  Fig 7
atggatttcctggaggaaccaataccaggtgt